(19) United States
(12) Patent Application Publication
(10) Pub. No.: US 2025/0268941 A1
(43) Pub. Date: Aug. 28, 2025

(54) BCMA/CD19 CAR FOR TREATING MULTIPLE MYELOMA

(71) Applicants: AUTOLUS LIMITED, London (GB); UCL BUSINESS LTD, London (GB)

(72) Inventors: Martin Pulé, London (GB); Lydia Lee, London (GB); Kwee Yong, London (GB); Simon Thomas, London (GB)

(21) Appl. No.: 18/967,332

(22) Filed: Dec. 3, 2024

Related U.S. Application Data
(63) Continuation of application No. 18/628,079, filed on Apr. 5, 2024, now abandoned.
Application No. 18/628,079 is a continuation of application No. 18/334,972, filed on Jun. 14, 2023, now abandoned.
(60) Provisional application No. 63/352,550, filed on Jun. 15, 2022.

(51) Int. Cl. A61K 35/17; A61K 9/00; A61K 40/11; A61K 40/31; A61K 40/42; A61P 35/00; C07K 16/28

(52) U.S. Cl. CPC A61K 35/17; A61K 9/0019; A61K 40/11; A61K 40/31; A61K 40/4211; A61K 40/4224; A61P 35/00; C07K 16/2803; C07K 16/2878; A61K 2239/13; A61K 2239/29; A61K 2239/31; A61K 2239/38; A61K 2239/48

(57) ABSTRACT

The present disclosure relates to BCMA/CD19 CAR T-cell products and methods for treating relapsed or refractory BCMA+ or CD19+ malignancies.

### CROSS-REFERENCE TO RELATED APPLICATIONS

This application is a Continuation of U.S. application Ser. No. 18/628,079, filed Apr. 5, 2025, which is a Continuation of U.S. application Ser. No. 18/334,972, filed Jun. 14, 2023, claims priority to U.S. Provisional Application No. 63/352,550, filed Jun. 15, 2022, which are incorporated herein by reference in their entirety.

The disclosure relates to BCMA/CD19 CAR T-cell products and methods for treating multiple myeloma.

### INCORPORATION BY REFERENCE OF THE SEQUENCE LISTING

This application contains, as a separate part of disclosure, a Sequence Listing in computer-readable form (Filename: 58127B_SeqListing.xml; 102,898 bytes—XML file dated Dec. 31, 2024) which is incorporated by reference herein in its entirety.

### BACKGROUND

Multiple myeloma (MM) is a plasma cell malignancy. Annually, nearly 6,000 patients are diagnosed with MM in the UK (https-//www.cancerresearchuk.org/health-professional/cancer-statis-tics/statistics-by-cancer-type/myeloma) placing MM within the top 20 commonest cancers nationally. The median age of diagnosis is 70-75 but the age distribution is wide and includes younger patients [Jurczyszyn et al., Leuk. Lymphoma, 60(2):471-476 (2019)]. Overall, 52% and 29% of patients survive more than 5 and 10 years respectively. Despite therapeutic advances, no treatment is curative and patients suffer repeated relapses with progressively shorter periods of time between each relapse, increasing disease resistance to standard therapies, burden of disease and treatment toxicities and eventual death [Yong et al., British Journal of Haematology, 175(2):252-264 (2016)]. Over 3,000 myeloma patients die a year in the UK due to their disease or from treatment related complications.

In the UK, standard treatment includes high dose chemotherapy and autologous haemato-poietic stem cell (HSC) rescue for younger patients who lack significant morbidities. For all patients, treatment centres around the three major drug classes: proteasome inhibitors (bortezomib, carfilzomib, ixazomib) and immunomodulatory drugs (IMiDs, thalidomide, lenalidomide, pomalidomide), with CD38-targeting antibodies (daratumumab and isatuximab) being the third major class.

Around 15-20% of patients respond poorly to current effective multi-drug regimens with refractory disease or early relapses, and all patients eventually become refractory to the major drug classes [Gandhi et al., Blood, 132.3233 (2018)].

In the UK, patients at 4^{th} line therapy have limited treatment options—typically, PomDex (pomalidomide, dexamethasone), or IsaPomDex (isatuximab, pomalidomide, dexamethasone) at fourth line and VDPano [Velcade (bortezomib), Daratumumab, panobinostat) at 5^{th} line. Pomalidomide and dexamethasone is often used as fourth line therapy and in the MM-003 trial, PFS was 4 months [Miguel et al., Lancet Oncol., 14(11):1055-1066 (2013)]. In a recent subgroup analysis of the ICARIA-MM study comparing outcomes of patients randomized to IsaPomDex or PD, PFS for patients at 4^{th} line therapy was 8.5 and 3.3 months respectively [Bringhen et al., Leukemia Research, 104:106576 (2021)]. VDPano was reported to have a PFS of 5 months in patients with a median 4 prior lines of therapy [Richardson et al., Blood, 122(14):2331-2337 (2013)] and, at UCLH, it has been reported a worse PFS of 3.5 months in a real-world and fifth line setting [Bird et al., Br J Haematol., 191(5):927-930 (2020)].

As myeloma patients progress through treatment lines, disease will become refractory to more therapeutic agents. In the UK, patients at fourth line therapy would have been ex-posed to PIs, IMiDs and, increasingly, CD38 antibodies and likely have disease refractory to these three major classes of antimyeloma therapy, so-called “triple refractory”. Patients with triple refractory disease have an estimated PFS of 4-5 months, with an estimated median overall survival (OS) of around 9 months [Kumar et al., Leukemia, 31(11):2443-2448 (2017); Usmani et al., Oncologist, 21(11):1355-1361 (2016)]. Thus, patients with disease refractory to conventional therapy have few licensed therapies available and outlook is poor. New approaches for these patients, particularly those which result in sustained responses, are needed.

### Chimeric Antigen Receptors (CARs)

Chimeric antigen receptors are proteins which graft the specificity of, for example, a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Successful CAR treatment depends on expression of the target antigen by tumour cells. In heterogenic tumours, in particular solid cancers, antigen expression is heterogeneous, and it may not be possible to find a single target antigen expressed by all cancer cells.

Moreover, emerging data from CAR T-cell trials in B-cell malignancies demonstrate that a common mechanism of resistance to this class of therapeutics is the emergence of tumours with loss or downregulation of the target antigen. Antigen loss or antigen-low escape is likely to emerge as an even greater barrier to success in solid tumours, which manifest greater heterogeneity in target antigen expression. Potential approaches to overcome this challenge include engineering CAR T cells to achieve multi-specificity and to respond to lower levels of target antigen and more efficient induction of natural antitumor immune responses as a result of CAR-induced inflammation.

Clinical studies of CAR T-cells have established that CAR T-cell engraftment, expansion and persistence are a pre-requisite for clinical activity, particularly sustained responses. A key reason for poor persistence of CAR-T cells in vivo, particularly CAR-T cells for the treatment of solid cancers, is that the cells struggle to overcome the hostile microenvironment of the tumour. In particular, CAR T-cells may fail to engraft and expand within a solid cancer tumour bed.

CAR T-cell persistence and activity can be enhanced by administration of cytokines, or by engineering the CAR T-cell to secrete or express cytokine, toxins or other factors. However, these approaches have limitations: systemic administration of cytokines can be toxic; constitutive production of cytokines may lead to uncontrolled proliferation and transformation.

### The BCMA Antigen

The restricted expression of BCMA [Bossen & Schneider, Semin Immunol., 18(5):263-275 (2006)] to plasma cells and plasmacytoid dendritic cells (pDCs) [Tai et al., Blood, 123-128-138 (2014)] makes it an attractive target for plasma cell directed therapies. Considerable clinical experience indicates that the hypogammaglobulinemia consequent to plasma cell depletion can be managed by administration of pooled immunoglobulin if clinically indicated. pDCs can detect pathogen-derived nucleic acids and studies in murine models would suggest that their depletion may contribute to increased risk of viral infections. However, given the redundancy in the function of dendritic cell subsets, and that other subsets of dendritic cells would be spared, clinically significant sequelae of pDCs are not expected.

Thus, BCMA has become the most promising therapeutic target for multiple myeloma. Indeed, BCMA targeting therapies appear well tolerated and efficacious even in relapsed and refractory disease. BCMA has been targeted as an antibody drug conjugate, CAR as well as Bispecific T cell engager.

### BCMA Directed CAR T Cell Therapies

BCMA CAR T cell therapy was first suggested and explored by Kochenderfer [Carpenter et al., Clin. Cancer Res., 19(8): 2048-2060 (2013)]. The first clinical study performed at the NIH used a CD28-CD3z CAR and showed efficacy, although with considerable immunotoxicity [Ali et al., Blood, 128(13):1688-1700 (2016); Brudno et al., Journal of Clinical Oncology, 36(22):2267-2280 (2018)]. Subsequently, multiple BCMA CAR T cells have been tested in clinical studies. Two BCMA CAR T cell therapies: Idecabtagene vicleucel (Ide-cel) and Ciltacabtagene autoleucel (cilta-cel) have progressed to phase II studies [Mikkilineni & Kochenderfer, Nat. Rev. Clin. Oncol., 18(2):71-84 (2021)]. Notably, both Ide-cel and Cilta-cel are lentivirally transduced autologous CAR T cell products with second-generation CARs with 4-1BB-CD3z endodomains.

The Ide-cel CAR comprises of an anti-BCMA scFv, CD8 spacer and a 41BB-CD3z endodomain manufactured by lentiviral transduction of autologous T cells. In a phase 2 trial, 128 patients were treated at 150/300/450×10⁶ CAR T cells [Munshi et al., New England Journal of Medicine, 384(8):705-716 (2021)]. Patients were heav-ily pretreated (6 median lines of therapy). The overall response in this Phase 2 trial of BB2121 was 73% with 33% of patients achieving a CR. The median duration of response was 8.8 months across these dose levels and 12.1 months at the highest dose level. Eighty-nine percent of patients experienced grade 3 or 4 cytopenias and while 84% of patients experienced CRS only 5% experienced grade 3 CRS. Eighteen percent of patients experienced neurological toxicity, but this was mostly mild with 3% of patients having grade 3-4 immune effector cell-associated neurotoxicity syndrome (ICANS). This product was licensed by the FDA in April 2021.

### Dual CD19 & BCMA CAR T Cells Targeting in Myeloma

By high resolution methods, there is CD19 expression on a significant proportion of tumour cells from the majority of patients, and at a sufficient density to activate CD19 CAR T cells. Thus targeting CD19 in the context of myeloma therapy may result in greater efficacy and durable responses. Garfall et al. conducted a pilot clinical trial (NCT02135406) targeted at patients with an expected progression free survival of less than 1 year after their first stem cell trans-plant [Garfall et al., JCI Insight. 21; 4(4) (2019).]. Ten MM patients were recruited and were treated with a combination of high-dose melphalan ASCT, and 1-5×10⁷ autologous CD19 CAR T cells (administered −2 weeks post-ASCT). Disease was not assessed pre ASCT and 6 out of 10 patients experi-enced a very good partial response (VGPR) at day 100 post-transplant, and an additional two patients had a PR. Two of the 10 patients experienced a longer PFS after 2nd transplant compared to first ASCT. Most adverse events were attributed to high dose melphalan (mu-cositis, haem toxicity, infections) and one of the patients experienced grade one CRS and there were no reports of neurotoxicity.

To date, there have been three main approaches to creating dual-targeting CARs. These strategies have included the mixing of 2 populations of CAR-transduced T cells, engineering a single CAR construct containing 2 separate scFvs in tandem (TanCAR) or the coexpression of 2 CARs on T cells using a bicistronic vector or double transduction (OR gate).

There is some early experience with CD19/BCMA CAR T cells summarised in Table 1. Thus far, the majority of CARs have been based on the co- or sequential administration of two different CAR products with one report of the dual transduction of CAR T cells with a tandem CAR construct expressing both BCMA and CD19 binding domains [Jiang et al., ASH (2020), https-//ash.confex.com/ash/2020/webprogram/Paper138614.html]. From this limited data, it would seem that although efficacy is at least equivocal to targeting BCMA alone (80-100%), rates of severe (≥Grade 3) CRS or neurotoxicity does not exceed targeting BCMA alone. Of note, the trial by Yan et al [(Yan et al., Cancer Medicine, 10(2):563-574 (2021)] observed higher rates of severe CRS than is typical while also using exceptionally high doses of T cells.

TABLE 1

| Trial | n | Dose | ORR | PFS | CRS | ICANS |
|---|---|---|---|---|---|---|
| Yan et al., The Lancet | 21 | BCMA CAR: 1 × 10⁶/kg | 95% | NR at | any - | any - |
| Haematology, | | CD19 CAR: 1 × 10⁶/kg | | 176 d | 90% ≥ gd | 10% ≥ gd |
| 6(10): e521-9 (2019). | | Both infused day 0 | | | 3-5% | 3-5% |
| Garfall et al., Blood. | 10 | 5 × 10⁸ BCMA and CD19 | 80% | NA | any - | any - |
| 2019 | | CAR T cells adminis- | | | 80% ≥ gd | 0% ≥ gd |
| 134(Suppl.1): 1863. | | tered in 3 doses (10%, | | | 3-0% | 3-0% |
| | | 30%, 60% | | | | |
| Shi et al. Blood, | 32 | Co-admin of anti- | 100% | 85% | any - | any - |
| 134(Suppl._1): 1949 | | CD19/BCMA-CD28- | | PFS at | 97% ≥ gd | 0% ≥ gd |
| (2019) | | OX40-ζ endodomain | | 1 year | 3-3% | 3-0% |
| | | CARS D14-20 after | | | | |
| | | ASCT 1 × 10⁷/kg sequen- | | | | |
| | | tially on day 0 and day 1 | | | | |
| Yan et al., Cancer | 9 | CD19 CAR: 1 × 10⁷/kg on | 100% | months | any - | any - |
| Medicine. | | day 0; BCMA CAR: | | | 100% ≥ gd | 4% ≥ gd |
| 2021; 10(2): 563-74 | | 2-6.8 × 10⁷/kg, 40% on day | | | 3-25% | 3-4% |
| | | 1, 60% on day 2 | | | | |
| Jiang et al, ASH; 2020 | 16 | CARS co-expressing | 94% | NA | any - | any - |
| [cited 2020 Dec. 28]. | | BCMA and CD19: | | | 88% ≥ gd | 0 ≥ gd |
| Available from: | | 1-3 × 10⁵/kg | | | 3-13% | 3-0% |
| https://ash.confex.com/ | | | | | | |
| ash/2020/webprogram/ | | | | | | |
| Paper138614.html | | | | | | |

Tumour heterogeneity describes the observation that different tumour cells can show distinct morphological and phenotypic profiles, including cellular morphology, gene expression, metabolism, motility, proliferation, and metastatic potential.

Heterogeneity occurs between patients, between tumours (inter-tumour heterogeneity) and within tumours (intra-tumour heterogeneity). Multiple types of heterogeneity have been observed between tumour cells, stemming from both genetic and non-genetic variability.

Heterogeneity between tumour cells can be further increased due to heterogeneity in the tumour microenvironment. Regional differences in the tumour (e.g. availability of oxygen) impose different selective pressures on tumour cells, leading to a wider spectrum of dominant subclones in different spatial regions of the tumour. The influence of microenvironment on clonal dominance is also a likely reason for the heterogeneity between primary and metastatic tumours seen in many patients, as well as the inter-tumour heterogeneity observed between patients with the same tumour type.

The heterogeneity of cancer cells introduces significant challenges in designing effective treatment strategies.

For example, heterogeneic tumours may exhibit different sensitivities to cytotoxic drugs among different clonal populations. This is attributed to clonal interactions that may inhibit or alter therapeutic efficacy.

Drug administration in heterogeneic tumours will seldom kill all tumour cells. The initial heterogeneic tumour population may bottleneck, such that few drug resistant cells (if any) will survive. This allows resistant tumour populations to replicate and grow a new tumour through the branching evolution mechanism (see above). The resulting repopulated tumour is heterogeneic and resistant to the initial drug therapy used. The repopulated tumour may also return in a more aggressive manner.

There is thus a need for alternative CAR treatment approaches which address the problems commonly encountered with CAR-T cell therapy, particularly bearing in mind the heterogeneity between patients, and between tumour cells and tumour cell sites within the same patient.

### SUMMARY

The disclosure provides methods for treating a refractory or relapsed BCMA+ or CD19+ malignancy in a patient comprising administering to the patient autologous BCMA/CD19 CAR T-cells (for example, the autologous BCMA/CD19 CAR T-cell product comprising CAT19CAR and 9A8CAR CARs described in Example 3 herein).

Methods are provided wherein the age of the patient is eighteen years or older.

Methods are provided wherein the BCMA+ or CD19+ malignancy is multiple myeloma (MM) or a BCMA+ or CD19+ plasma cell disorder. Methods are provided wherein the BCMA+ or CD19+ malignancy is multiple myeloma (MM).

Methods are provided in particular wherein the patient has: a) Relapsed or refractory Multiple Myeloma; b) Secretory disease; c) 23 prior lines of therapy; d) Refractory to last line of therapy; or e) Has previously received or is not suitable for autologous stem cell transplant (ASCT); In the methods provided, the patient is administered a single dose of 5×10⁶ CAR T-cells, 25×10⁶ CAR T-cells, 50×10⁶ CAR T-cells, or 150×10⁶ CAR T-cells. The administration may be an intravenous injection through a Hickman line or peripherally inserted central catheter.

Methods are provided wherein the BCMA/CD19 CAR T-cells express a chimeric antigen receptor (CAR) comprising a BCMA-binding domain which comprises
a) a heavy chain variable region (VH) having CDRs with the following sequences:

```
  (SEQ ID NO: 1)
CDR1-GFIFSDYN
  (SEQ ID NO: 2)
CDR2-IIYDGSST
  (SEQ ID NO: 3)
CDR3-ATRPGPFAY;
```

b) a light chain variable region (VL) having CDRs with the following sequences:

```
  (SEQ ID NO: 4)
CDR1-QSLLHSNGNTY
  (SEQ ID NO: 5)
CDR2-LVS
(SEQ ID NO: 6)
CDR3-VHGTHAWT.
```

The CDRs may be grafted on to a human antibody framework.

In provided methods, the BCMA-binding domain comprises a VH domain having the sequence shown as SEQ ID NO: 7 and/or or a VL domain having the sequence shown as SEQ ID NO: 8 or a variant thereof having at least 95% sequence identity.

The BCMA-binding domain may comprise an scFv in the orientation VH-VL or VL-VH.

The BCMA-binding domain may comprise a Fab. The BCMA-binding Fab may comprise a heavy chain (VH-CH1) sequence shown as SEQ ID NO: 9. The BCMA-binding Fab may comprise a light chain (VL-CL kappa) sequence shown as SEQ ID NO: 10.

The BCMA-binding domain and a transmembrane domain may be connected in the CAR by a spacer such as a human IgG1 hinge. The CAR may comprise intracellular T cell signaling domain such as an intracellular T-cell signaling domain comprising the 41BB endodomain and the CD3-Zeta endodomain.

Methods are provided wherein the BCMA/CD19 CAR T-cells express a chimeric antigen receptor (CAR) comprising a CD19-binding domain which comprises
a) a heavy chain variable region (VH) having complementarity determining regions (CDRs) with the following sequences:

```
  (SEQ ID NO: 37)
CDR1-GYAFSSS;
  (SEQ ID NO: 38)
CDR2-YPGDED
  (SEQ ID NO: 39)
CDR3-SLLYGDYLDY;
```

b) a light chain variable region (VL) having CDRs with the following sequences:

```
  (SEQ ID NO: 40)
CDR1-SASSSVSYMH;
  (SEQ ID NO: 41)
CDR2-DTSKLAS
(SEQ ID NO: 42)
CDR3-QQWNINPLT.
```

The CDRs may be grafted on to a human antibody framework.

In provided methods, the CD19-binding domain may comprise a VH domain having the sequence shown as SEQ ID NO: 43 and/or or a VL domain having the sequence shown as SEQ ID NO: 44, or a variant of either thereof having at least 95% sequence identity.

The CD19-binding domain may comprise an scFv in the orientation VH-VL. The CD19-binding domain may comprise the sequence shown as SEQ ID NO: 45 or a variant thereof having at least 90% sequence identity. The CD19-binding domain and a transmembrane domain may be connected in the CAR by a spacer such as a CD8 stalk. The CAR may comprise intracellular T cell signaling domain such as an intracellular T-cell signaling domain comprising the 41BB endodomain and the CD3-Zeta endodomain.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1—Schematic diagram showing a classical chimeric antigen receptors (a) Basic schema of a chimeric antigen receptor; (b) First generation receptors; (c) Second generation receptors; (d) Third generation receptors.

FIG. 2—Different binding domain formats of chimeric antigen receptors. (a) Fab CAR format; (b) dAb CAR format; (c) scFv CAR format

FIG. 3—Ficolled BM MNCs from 50 patients were stained for CD138 to identify tumor and anti-BCMA by using QuantiBRITE beads for antigen quantification. We found expression of BCMA on CD138 tumor cells ranged from 105-8323 antigens per cell with a median of 1061 [Lee et al., Blood. 2018; 131(7):746-758 (2018)].

FIG. 4—Functional screening of different BCMA binders against SupT1, SupT1.BCMAlow and JeKo-1 cells (1:4 effector: target ratio for 24 hours. Shown are the results of 9 independent donors). (Please note, the higher IL-2 produced against JeKo-1 cells despite lower BCMA density is expected since JeKo-1 cells express co-stimulatory ligands and SupT1 cells do not).

FIG. 5—Flow-cytometric determination of D8 IgG binding to cell membrane bound BCMA. SupT1 is a T cell line which does not express BCMA. As stated previously, SupT1.BCMAhigh and SupT1.BCMAlow cells were engineered to express BCMA at high and low levels respectively. MM1.s cell line is a B lymphoblast cell line expressing BCMA at intermediate levels. Each cell line was stained with either IgG and a secondary fluorescent anti-Fc antibody, or the secondary antibody alone.

FIG. 6—Surface plasmon resonance sensogram of binding kinetics between D8 IgG and BCMA is shown. BCMA starting concentration was 50 nM with serial dilutions to 0.19 nM. Kinetic affinity, expressed as KD (M), was measured at 0.126 nM for D8 IgG with kd and ka of 3.45×10\hat{ }(1/Ms) and 4.33×10-3 (1/s) respectively.

FIG. 7—(a)D8-41BB-ζ Architecture is a standard CAR. Binding domain is in a scFv format with CD8 stalk as a spacer. This is expressed as a single protein. (b)D8Fab-41BB-ζ architecture is in FabCAR format. Here, two separate proteins comprise the CAR with the VL andKappa common chain as one protein and VH, CH1 and signaling endodomains as the second protein. Both are expressed from a single open reading frame but translated as two separate proteins by use of a foot-and-mouth 2A-like peptide (denoted by scissors).

FIG. 8—Surface expression of D8-41BB-ζ and D8Fab-41BB-ζ on primary human T cells. (a) Example flow cytometry plot is shown with marker gene staining (RQR8) on the X-axis and staining with recombinant BCMA-Fc on the Y-axis. (b) Median Fluorescent intensity of BCMA staining from 6 donors.

FIG. 9—(a) IL-2 release of D8 CARs in scFv or Fab format in response to different target cells. (b) and (c) IL-2 and IFN− response to increasing concentrations of plate bound BCMA; (d) and (e) CD69 expression in CD8+ and CD8− CAR T cells determined by flow-cytometry in response to increasing concentrations of plate-bound BCMA.

FIG. 10—Cytotoxic effects of D8 CAR T cells In Vitro. D8 CAR T cells were co-cultured with SupT1 NT, SupT1 BCMAlow, JeKo-1 and MM.1s cells at an effector:target ratio of 1:8 for 24 hours before supernatants were collected and analysed for cytokine production, and target cells were enumerated. Mean±SD shown; two-tailed paired T tests were used, ns=not significant, *p<0.05, **p<0.01, **p<0.001, **p<0.0001; n=6. Note the differences in y-axis scaling between graphs.

FIG. 11—Cytokine production by D8 CAR T cells In Vitro. D8 CAR T cells were co-cultured with SupT1 NT, SupT1 BCMAlow, JeKo-1 and MM.1s cells at an effector:target ratio of 1:8 for 24 hours before supernatants were collected and analysed for cytokine production. TOP: IFN-γ levels from the 24-hour co-cultures. BOTTOM: IL-2 levels from the 24-hour co-cultures. Mean±SD shown; two-tailed paired T tests were used, ns=not significant, *p<0.05, *p<0.01, ***p<0.001, ****p<0.0001; n=6. Note the differences in y-axis scaling between graphs.

FIG. 12—Proliferation of BCMA-CAR T Cells In Vitro. Proliferation of BCMA-AR T cells were assessed by co-culturing CTV-labelled CAR T cells with BCMA-expressing targets at an effector:target ratio of 1:1 and T cells were labelled with CTV prior to co-culture. Co-cultures were acquired and analysed for CTV dilution after 96 hours. CAR T cells were gated based on CD3+BCMA-Fc+ expression and divided into CD8− or CD8+ T cell populations. (A) Representative histograms of CTV dilution from one donor. (B) CTV-diluted CAR T cells (subdivided into CD8+ and CD8− populations) were gated as % proliferated cells. Mean±SD shown; two-way ANOVA with Tukey's test was used, *p<0.05, *p<0.01, ****p<0.0001; n=6.

FIG. 13—Cytotoxicity and cytokine production of D8, bb2121 and LCAR-B38M CAR T cells against BCMA-expressing targets in vitro. D8, bb2121 and LCAR-B38M CAR T cells were co-cultured with SupT1 NT, SupT1 BCMAlow, JeKo-1 and MM.1s cells at an effector:target ratio of 1:8 for 24 hours, before target cells were enumerated for as a measure of CAR cytotoxicity. (A) Viable targets recovered from co-cultures normalised to that recovered from co-cultures with mock-transduced (NT) T cells (%). (B) Culture supernatants from these 24-hour co-cultures were analysed for (left) IFNγ and (right) IL-2 production. Mean±SD shown; one-way ANOVA with post hoc Tukey's test used, *p<0.05, *p<0.01, **p<0.001; n=8.

FIG. 14—Activation of D8 BCMA CAR, bb2121 CAR and LCAR-B38M transduced T cells against plate-bound BCMA antigen stimulation. (A) D8 BCMA CAR, bb2121 or LCAR-B38M BCMA-CAR-transduced T cells were plated with increasing concentrations of plate-bound BCMA for 24 hours before analysed by flow cytometry for CD69 expression. Expression of these activation markers were analysed as median fluorescence intensity (MFI) for both CD8+ and CD8− CAR T cell populations. Mean±SD shown; non-linear regression analyses were conducted ([agonist] vs response−variable slope (four parameter)); n=6. (B) Culture supernatant were collected at the 24-hour time point and analysed for (left) IFNγ and (right) IL-2 production by ELISA. Mean±SD shown; non-linear regression analyses were conducted ([agonist] vs response −variable slope (four parameter)); n=6.

FIG. 15—(a) Double transduction of normal donor T cells with separate vectors encoding D8Fab-41BB-ζ and CAT-41BB-ζ. CAR expression on live CD3+ cells were detected by staining with CAT19 anti-idiotype plus anti-Rat-Fc-PE for detection of CAT-41BB-ζ, and with BCMA-AviTag+streptavidin-APC for detection of D8Fab-41BB-ζ. Flow cytometry plots from a representative transduction are shown, with mock-transduced (NT) control, D8 single transduction, CAT single transduction, and D8/CAT dual transduction. (b) Right graph shows percentages of D8−CAT−, D8+CAT−, DB+CAT+ and D8−CAT+ cells within the CD3+ population of the transduced PBMCs for 9 donors, with mean±SD shown.

FIG. 16—Cytotoxicity and cytokine production of D8, CAT and D8/CAT CAR T cells against target cells which express neither, one or both BCMA and CD19. A) For cytotoxicity assessment CAR-transduced T cells were co-cultured with antigen-negative SupT1 NT, SupT1.BCMAlow, SupT1.CD19 or SupT1 BCMAlow.CD19 targets at effector-target ratio of 1:1 for 96 hours, before target cells were enumerated for as a measure of CAR cytotoxicity. Viable targets recovered from co-cultures with CAR-transduced T cells were normalised to that recovered from co-cultures with mock-transduced (NT) T cells (%). Mean±SD shown; Two-way ANOVA with post hoc Dunnett's test was used, *p<0.01, ***p<0.001, ns=not significant; n=6. B) For measurement of cytokine production CAR-Transduced T cells were co-cultured with the indicated target cells for 24 hours at an effector target ratio of 1:4.

FIG. 17—Proliferation of D8, CAT and D8/CAT CAR T cells in response to different targets expressing neither, one or both of target antigens BCMA and CD19. Proliferation of different CAR T cells were compared by co-culturing CellTrace Violet (CTV)-labelled CAR T cells with SupT1 cells, SupT1.BCMAlow, SupT1.CD19, SupT1.BCMAlow.CD19, JeKo-1 and MM.1s target cells at an effector-target ratio of 1:1 for 96 hours, before acquisition by flow cytometry and analysis of CTV dilution as a measure of proliferation. Note: JeKo-1 cells express both BCMA and CD19; MM.1s cells express BCMA, but not CD19. Proliferation of CD8+ or (bottom graph) CD8− CAR populations, where CTV-diluted CAR T cells were gated as proliferated cells and expressed as percentages (%). Mean±SD shown; Two-way ANOVA with post hoc Dunnett's test was used, *p<0.01*, p<0.001, ****p<0.0001, ns=not signifcant; n=6.

FIG. 18—Top panel, summary of experimental design, Bottom panel: bioluminescence of different cohorts (media alone, N=6; NT, n=5; D8 CAR. N=4; D8/CAT CAR, n=5) following injection with CAR T cells.

FIG. 19—Annotated amino acid sequence (SEQ ID NO: 93) of the CD19 CATCAR (AUTO 1).

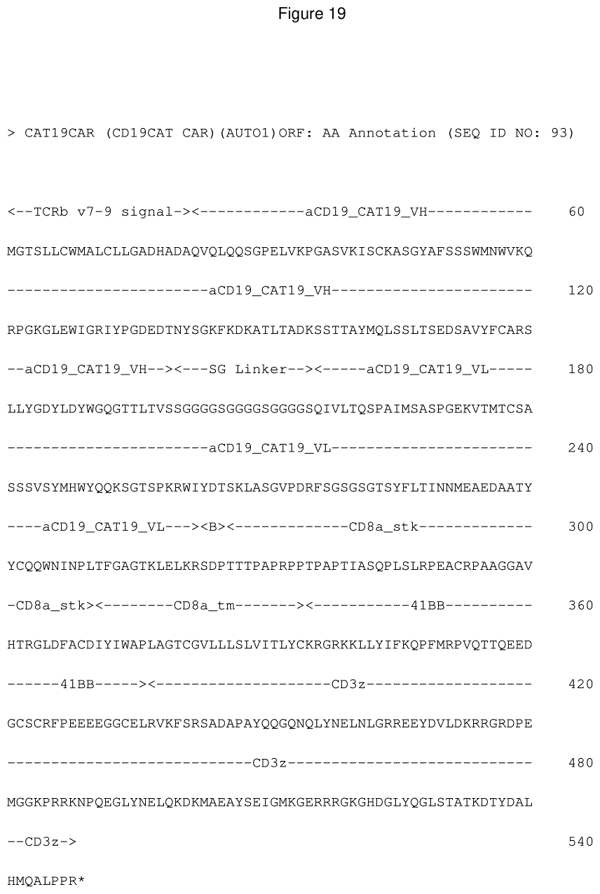

FIG. 20—Annotated amino acid sequence (SEQ ID NO: 94) of the BCMA CAR (D8 FabCAR).

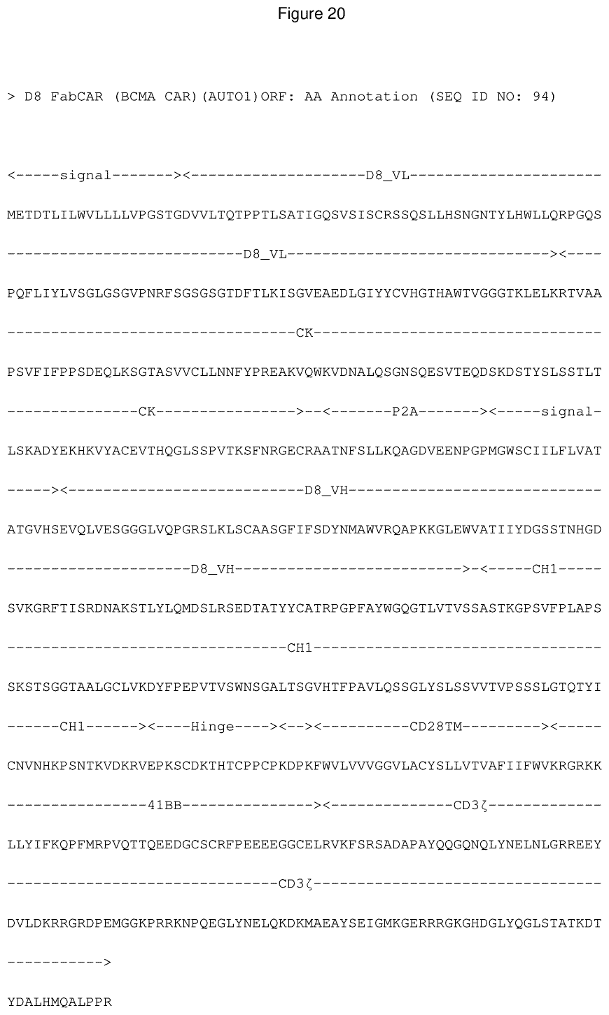

### DETAILED DESCRIPTION

In the KARMMA clinical study (Munshi et al., 2021, supra), which tested an anti-BCMA scFv, CD8 spacer and a 41BB-CD3z endo-domain (BB2121), the overall response in this Phase 2 trial of BB2121 was 73% with 33% of patients achieving a CR. The median duration of response was 8.8 months across these dose levels and 12.1 months at the highest dose level. Thus, unlike CD19 CAR T cell therapy, BCMA CAR T cells may not however result in long term remissions. There may be several reasons for this—firstly BCMA CAR T cells do not persist in vivo for as long as CD19 CAR T cells. This may be because BCMA antigen density is very low; additionally, the CD19 antigen is more ubiquitously expressed than BCMA providing more CAR T cell activation. Further, relapse may be due to a MM stem cell, which may be a late-stage B cell which does not express BCMA; and in some cases, BCMA may be lost by MM cells.

CD19 is contemplated herein as another target for MM. Targeting BCMA and CD19 simultaneously may prevent relapse by depleting MM stem cells, pre-venting antigen escape, and CD19 co-targeting may result in longer CAR T cell persistence.

The methods provided herein improve the treatment of r/r MM by combining a highly sensitive BCMA CAR capable of targeting cells that express around 100 BCMA molecules per cell (BCMA CAR or D8 CAR), with AUTO1 (CD19 CAR, CATCAR), which is an anti-CD19 CAR having a fast off-rate, to generate a dual targeting CD19 and BCMA product via co-transduction for the treatment of pediatric r/r MM. Results described in the Examples below were obtained as part of the MCARTY clinical trial (NCT04795882).

### Chimeric Antigen Receptors (CARs)

A classical chimeric antigen receptor (CAR) is a chimeric type I trans-membrane protein which connects an extracellular antigen-binding domain to an intracellular signalling domain (endodomain). The antigen-binding domain is typically a single-chain variable fragment (scFv) derived from a monoclonal antibody (mAb), but it can be based on other formats which comprise an antibody fragment or an antibody-like antigen-binding site. Other examples include, but are not limited to: a natural ligand of the target antigen, a peptide with sufficient affinity for the target, a F(ab) fragment, a F(ab′)₂ fragment, a F(ab′) fragment, a single domain antibody (sdAb), a domain antibody (dAb), a VHH antigen-binding domain or nanobody, an artificial single binder such as a DARPin (designed ankyrin repeat protein), an affibody, a fibronectin artificial antibody scaffold, an anticalin, an affilin, a VNAR, an iBody, an affimer, a fynomer, an abdurin/nanoantibody, a centyrin, an alphabody, a nanofitin, or a single-chain derived from a T-cell receptor which is capable of binding the target antigen. A spacer is usually necessary to isolate the antigen-binding domain from the membrane and to allow it a suitable orientation. A common spacer used is the Fc of IgG1. More compact spacers can suffice, e.g., the stalk from CD8a and even just the IgG1 hinge alone, depending on the antigen. A transmembrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. One common co-stimulatory domain is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target antigen, an activating signal is transmitted to the T-cell on which the CAR is expressed thereby directing the specificity and cytotoxicity of the T cell towards cells expressing the target antigen. CAR-encoding nucleic acids may be transferred to T cells using, for example, retroviral or lentiviral vectors to generate cancer-specific T cells for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

### Antigen Binding Domain

The antigen binding domain is the portion of CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

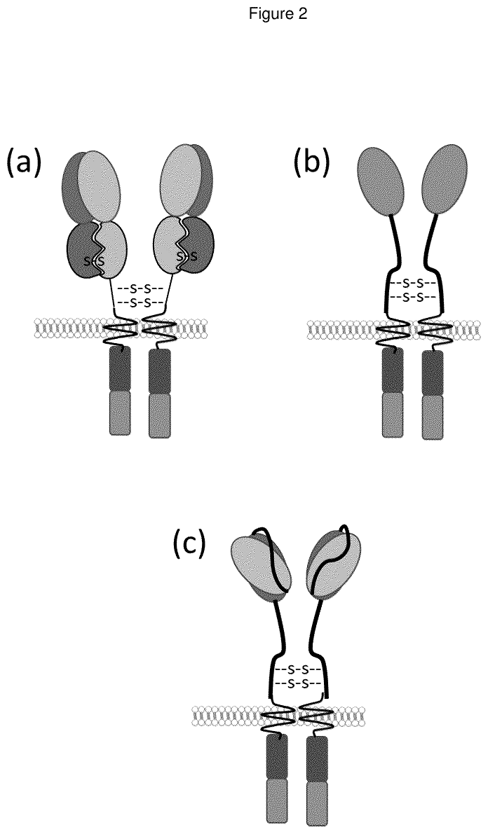

In a classical CAR, the antigen-binding domain comprises: a single-chain variable fragment (scFv) derived from a monoclonal antibody (see FIG. 2c). CARs have also been produced with domain antibody (dAb) or VHH antigen binding domains (see FIG. 2b) or which comprise a Fab fragment of, for example, a monoclonal antibody (see FIG. 2a). A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The two chains of a Fab CAR may have the general structure:
VH-CH-spacer-transmembrane domain-intracellular signalling domain; and
VL-CL
or
VL-CL-spacer-transmembrane domain-intracellular signalling domain; and
VH-CH

For Fab-type chimeric receptors, the antigen binding domain is made up of a VH from one polypeptide chain and a VL from another polypeptide chain.

The polypeptide chains may comprise a linker between the VHNL domain and the CH/CL domains. The linker may be flexible and serve to spatially separate the VHNL domain from the CH/CL domain.

### Target Antigens

A ‘target antigen’ is an entity which is specifically recognized and bound by the antigen-binding domains of a chimeric receptor provided herein.

The target antigen may be an antigen present on a cancer cell, for example, a tumor-associated antigen. BCMA and CD19 are target antigens contemplated herein.

### Binding Domains Specific for BCMA Target Antigen

The B cell maturation target, also known as BCMA; TR17_HUMAN, TNFRSF17 (UniProt Accession No. 002223, entry version 200, https-//www.uniprot.org/uniprot/Q02223) is a transmembrane protein that is expressed in mature lymphocytes, e.g., memory B cells, plasmablasts and bone marrow plasma cells. BCMA is also expressed on myeloma cells. BCMA is a non-glycosylated type Ill transmembrane protein, which is involved in B cell maturation, growth and survival.

An antigen binding domain of a CAR which binds to BCMA may be any domain which is capable of binding BCMA. The VH and VL sequences for fourteen anti-BCMA antibodies are given below with CDR sequences in bold and underlined.

The sequence of BCMA is depicted under UniProt Accession No. 002223, entry version 200 (https-//www.uniprot.org/uniprot/Q02223).

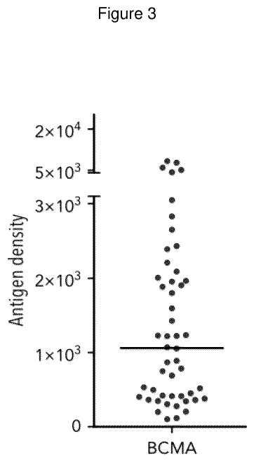

BCMA is an excellent MM target since it is expressed on practically all cases of MM and expression is otherwise restricted to normal plasma cells. However, it is a low-density antigen (FIG. 3), so a key consideration in selecting a BCMA CAR was sensitivity to low antigen density.

A number of BCMA-targeted CARs are in clinical development, including bb2121, LCAR-B38M, MCARH171, JCARH125, P-BCMA-101, FCARH143, bb21217 and CT053.

WO2015/052538 describes a BCMA targeted CAR in which the antigen-binding domain is derived from APRIL, which is a natural ligand for BCMA.

WO2020/065330, which is incorporated herein by reference, describes the VH and VL domains for 14 BCMA binding domains and their use in CARs.

The BCMA antigen-binding domain may comprise:
a) a heavy chain variable region (VH) having complementarity determining regions (CDRs) with the following sequences:

```
  (SEQ ID NO: 1)
CDR1-GFIFSDYN
  (SEQ ID NO: 2)
CDR2-IIYDGSST
  (SEQ ID NO: 3)
CDR3-ATRPGPFAY;
```

b) a light chain variable region (VL) having complementarity determining regions (CDRs) with the following sequences:

(SEQ ID NO: 4)
CDR1-QSLLHSNGNTY
(SEQ ID NO: 5)
CDR2-LVS
(SEQ ID NO: 6)
CDR3-VHGTHAWT

It is contemplated that it is possible to introduce one or more mutations (substitutions, additions or deletions) into one or more CDRs without negatively affecting BCMA-binding activity. Each CDR may, for example, have one, two or three amino acid mutations.

The BCMA antigen-binding domain may comprise the following VH domain

D8 VH domain
(SEQ ID NO: 7)
EVQLVESGGGLVQPGRSLKLSCAASGFIFSDYNMAWVRQAPKKGLEWVA
TIIYDGSSTNHGDSVKGRFTISRDNAKSTLYLQMDSLRSEDTATYYCAT
RPGPFAYWGQGTLVTVS

The BCMA antigen-binding domain may comprise the following VL domain

D8 VL domain
(SEQ ID NO: 8)
DVVLTQTPPTLSATIGQSVSISCRSSQSLLHSNGNTYLHWLLQRPGQSP
QFLIYLVSGLGSGVPNRFSGSGSGTDFTLKISGVEAEDLGIYYCVHGTH
AWTVGGGTKLELK

The BCMA antigen-binding domain may comprise an anti-BCMA Fab CAR.

The BCMA antigen-binding domain may comprise the following heavy chain (VH-CH1) sequence:

D8 heavy chain
(SEQ ID NO: 9)
EVQLVESGGGLVQPGRSLKLSCAASGFIFSDYNMAWVRQAPKKGLEWVA
TIIYDGSSTNHGDSVKGRFTISRDNAKSTLYLQMDSLRSEDTATYYCAT
RPGPFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKRV

The BCMA antigen-binding domain may comprise the following light chain (VL-Ckappa) sequence

D8 light chain
(SEQ ID NO: 10)
DVVLTQTPPTLSATIGQSVSISCRSSQSLLHSNGNTYLHWLLQRPGQSP
QFLIYLVSGLGSGVPNRFSGSGSGTDFTLKISGVEAEDLGIYYCVHGTH
AWTVGGGTKLELKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

The CAR may comprise the following sequences:

(CAR light chain: D8 VL-Ckappa)
SEQ ID NO: 11
DVVLTQTPPTLSATIGQSVSISCRSSQSLLHSNGNTYLHWLLQRPGQSPQFLIYLVS
GLGSGVPNRFSGSGSGTDFTLKISGVEAEDLGIYYCVHGTHAWTVGGGTKLELKRT
VAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
(CAR heavy chain: D8 VH-CH1-IgG1 hinge-CD28TM-41BB-CD3z)
SEQ ID NO: 12
EVQLVESGGGLVQPGRSLKLSCAASGFIFSDYNMAWVRQAPKKGLEWVATIIYDGS
STNHGDSVKGRFTISRDNAKSTLYLQMDSLRSEDTATYYCATRPGPFAYWGQGTLV
TVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTC
PPCPKDPKFWVLVVVGGVLACYSLLVTVAFIIFWVKRGRKKLLYIFKQPFMRPVQTT
QEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGL
YQGLSTATKDTYDALHMQALPPR

The CAR provided herein may comprise a variant of the sequence shown as any of SEQ ID NO: 1-12 having at least 80, 85, 90, 95, 98 or 99% sequence identity, provided that the variant sequence retain the capacity to bind BCMA (when in conjunction with a complementary VL or VH domain, if appropriate).

BCMA antigen-binding domain may be any domain which is capable of binding BCMA. The VH and VL sequences for twelve anti-BCMA antibodies are given below with CDR sequences in bold and underlined.

(anti-BCMA Ab1 VH)
SEQ ID NO: 13
QIQLVQSGPELVKPGSSVKLSCKTSGFTFSDSYMSWLKQVPGQSIEWIGNIYAGDG
ATHYHQKFKGKATLTVDTSSSTAYMDLSSLTSEDSALYFCARPLYTTAYYYVGGFA
YWGQGTLVTVSS
(anti-BCMA Ab1 VL)
SEQ ID NO: 14
DIVMTQSPSSLAVSAGETVTINCKSSQSLLSSGNQKNYLAWYQQKPGQSPKLLIYW
ASTRQSGVPDRFIGSGSGTDFTLTISSVQAEDLAIYYCQQYYDTPLTFGSGTKLEIK
(anti-BCMA Ab2 VH)
SEQ ID NO: 15
EVKLVESGGGLVQPGRSLKLSCTASGFTFSNYDMAWVRQAPTKGLEWVASISTSG
DTIYYRDSVKGRFTVSRDKAKSTLYLQMDSLRSEDTATYYCARHDYYDGYQSFAY
WGQGTLVTVSS
(anti-BCMA Ab2 VL)
SEQ ID NO: 16
NTVMTQSPTSMSISVGDRVTMNCKASQNVGNNIAWYQQKPGQSPKLLIYYASNRYT
GVPDRFTGSGSGTDFTLTINSVQAEDAAFYYCQRIYNSALTFGSGTKLEIK
(anti-BCMA Ab3 VH)
SEQ ID NO: 17
QVQLQQSGAALVKPGASVKMSCKASGYTFTDYWVSWVKQSHGKSLEWIGEIYPNS
GPTNFNKKFKGKATLTVDKSTSTAYMELSRLTSEDSAIYYCTPRTVAPYNWFAYWG
QGTLVTVSS
(anti-BCMA Ab3 VL)
SEQ ID NO: 18
DIVLTQSPALAVSPGERVSISCRASESVSTRMHWYQQKPGQQPKLLIYGASNLESG
VPARFSGSGSGTDFTLTIDPVEADDTATYFCQQSWNDPYTFGAGTKLELK
(anti-BCMA Ab4 VH)
SEQ ID NO: 19
EVQLVESGGGLVQPGRSLKLSCSASGFIFSNFDMAWVRQAPRKGLEWVASITTSG
GDTHYRDSVKGRFTVSRHNAKSTLYLQMDSLRSEDTATYYCARHVYYGLFWFFDF
WGPGTMVTVSS
(anti-BCMA Ab4 VL)
SEQ ID NO: 20
NTVMTQSPKSIFISVGDRVTVNCKASQNVGTNVDWYQQKTGQSPKLLIYGASNRYT
GVPDRFTGSGSGTDFTFTISNMQAEDLAVYYCMQSNTNPFTFGAGTKLELKR
(anti-BCMA Ab5 VH)
SEQ ID NO: 21
EVQLVESGGGLVQPGRSLKLSCTASGFTFSNYDMAWVRQAPTKGLEWVASISTSG
DTIYYRDSVKGRFTVSRDKAKSTLYLQMDSLRSEDTATYYCARHDYYDGYQSFAY
WGQGTLVTVSS
(anti-BCMA Ab5 VL)
SEQ ID NO: 22
DIVMTQSPSTLPASLGERVTISCRASQSISNYLNWYQQKPDGTIKPLIYYTSNLQSGV
PSRFSGSGSGTDYSLTISSLEPEDFAMYYCQQDASFPWTFGGGTKLELKR
(anti-BCMA Ab6 VH)
SEQ ID NO: 23
EVQLQESGPGLVKPSQSLSLTCSVTGYPITNNYDWSWIRQFPGNKMEWMGYISDS
GNTNYNPSLKSRISITRDTSKNQFFLQLNSVTTEDTATYYCASGYISYIPFAFWGQGT
LVTVSS
(anti-BCMA Ab6 VL)
SEQ ID NO: 24
DIVLTQSPALAVSLGQRATISCRASQSVSISSYNLMQWYQQKPGQQPKLLIYDASNL
ASGIPARFSGSGSGTDFTLTIDPVQADDIATYYCQQSKDDPNTFGAGTKLEIKR
(anti-BCMA Ab7 VH)
SEQ ID NO: 25
EVQLQESGPGLVQPSQTLSLTCSVTGYPITNNYDWSWIRKFPGNKMEWMGYISDS
GSTNYNPSLKSRISITRDTSKNQFFLQLNSVTTEDTATYYCASGYISYIPFGFWGQGT
LVTVSS
(anti-BCMA Ab7 VL)
SEQ ID NO: 26
DIVLTQSPALAVSPGERVTISCRASESVSTRMHWYQQKPGQQPKLLIYGASNLESG
VPARFSGSGSGTDFTLTIDPVEADDTATYFCQQSWNDPPTFGSGTKLEIK
(anti-BCMA Ab8 VH)
SEQ ID NO: 27
EVQLVESGGGLVQPGRSLKLSCTASGFTFSNYDMAWVRQAPTKGLEWVASISTSG
DTIYYRDSVKGRFTVSRDKAKSTLYLQMDSLRSEDTATYYCARHDYYDGYQSFAY
WGQGTLVTVSS
(anti-BCMA Ab8 VL)
SEQ ID NO: 28
DIVMTQSPASQAVSAGEKVTMSCKSSQSLLYSGDQKNYLAWYQQKPGQSPKLLIYL
ASTRESGVPDRFIGSGSGTDFTLTISSVQAEDLADYYCQQHYSYPLTFGSGTKLEIK
(anti-BCMA Ab9 VH)
SEQ ID NO: 29
EVQLVESGGGLVQPGRSLKLSCAASGFTFSNYDMAWVRQAPTKGLEWVASISTSG
DTIYYRDSVKGRFTVSRDNAKSTLYLQMDSLRSEDTATYYCTRHGYYDGYQSFDY
WGQGTLVTVSS
(anti-BCMA Ab9 VL)
SEQ ID NO: 30
NTVMTQSPKSMSISVGDRVTMNCKASQNVGNNIAWYQQKPGQSPKLLIYYASNRY
TGVPDRFTGGGYGTDFTLTINSVQAEDAATYYCQQWNYPSITFGSGTKLEIK
(anti-BCMA Ab10 VH)
SEQ ID NO: 31
EVQLVESGGGLVQPGRSMKLSCAASGFTFSNYDMAWVRQAPTKGLEWVASISPSG
GSTYYRDSVKGRFTVSRDNAKSSLYLQMDSLRSEDTATYYCTRGDYGYNYAYWFA
YWGQGTLVTVSS
(anti-BCMA Ab10 VL)
SEQ ID NO: 32
DIVMTQAPSSMPASLGERVTISCRASQGISNYLNWYQQKPDGTIKPLIYYTSNLQSG
VPSRFSGSGSGTDYSLTISSLEPEDFAMYYCQQYDSSPLTFGAGTKLELK
(anti-BCMA Ab11 VH)
SEQ ID NO: 33
EVQLVESGGGLVQPGRSLKLSCEASGFTFSNYDMAWVRQAPTKGLEWVASISTSG
DSIYYRDSVKGRFTVSRDNVKSTLYLQMDSLRSEDTATYYCARHGYYDGYQSFDY
WGQGTLVTVSS
(anti-BCMA Ab12 VL)
SEQ ID NO: 34
DIVMTQSPSSLPASLGERVTISCRASQGISNNLNWYQQKPDGTIKPLIYYTSNLQSGV
PSRFSGSGSGTDYSLTISSLEPEDFATYYCQQDETFPYTFGAGTKLELK
(anti-BCMA Ab13 VH)
SEQ ID NO: 35
EVQLVESGGGLVQPGRSLKLSCAASGFTFSNYDMAWVRQAPTKGLEWVASISPSG
GSTYYRDSVKGRFTISRDNAKSTLYLQMDSLRSEDTATYYCATHNYYDGSSLFAYW
GQGTLVTVSS
(anti-BCMA Ab13 VL)
SEQ ID NO: 36
DIVLTQSPALAVSPGERVTISCGANETVSTLVHWYQQKPGQQPKLLIYLASHLESGV
PARFSGSGSGTDFTLTIDPVEADDTATYYCQQSWNDPPTFGGGTKLELK

The percentage identity between two polypeptide sequences may be readily determined by programs such as BLAST which is freely available at blast.ncbi.nlm.nih.gov.

The BCMA binder may have a kinetic affinity (KD) of 10 nM or less, or 5 nM or less, or 1 nM or less, or 0.5 nM or less, or 0.1 nM or less.

### OR Gates

The CAR may be used in a combination with one or more other activating or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:
“OR GATE”—T cell triggers when either antigen A or antigen B is present on the target cell
“AND GATE”—T cell triggers only when both antigens A and B are present on the target cell
“AND NOT GATE”—T cell triggers if antigen A is present alone on the target cell, but not if both antigens A and B are present on the target cell Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A +antigen B. This is especially important for antigens expressed at variable or low density on the target cell, as the level of a single antigen may be below the threshold needed for effective targeting by a CAR-T cell. Also, it avoids the phenomenon of antigen escape. For example, some MM may become BCMA negative after BCMA targeting: using an OR gate which targets BCMA in combination with another antigen provides a “back-up” antigen, should this occur.

The OR gate may comprise a CAR against a second antigen expressed in MM cells, such as CD19.

The second CAR may have any suitable antigen binding domain, for example a binding domain based on an scFv, a domain antibody (dAb) or a Fab.

Thus, the antigen-binding domains of the first and second CARs bind to different antigens and both CARs may comprise an activating endodomain. The two CARs may comprise spacer domains which may be the same, or sufficiently different to prevent cross-pairing of the two different receptors. As contemplated herein a cell can hence be engineered to activate upon recognition of either or both BCMA and CD19.

This is useful in the field of oncology as indicated by the Goldie-Coldman hypothesis: sole targeting of a single antigen may result in tumor escape by modulation of said antigen due to the high mutation rate inherent in most cancers. By simultaneously targeting two antigens, the probably of such escape is exponentially reduced.

It is important that the two CARs do not heterodimerize.

The first and second CAR of the T cell may be produced as a polypeptide comprising both CARs, together with a cleavage site.

Binding domains specific for CD19 target antigen The human CD19 antigen is a 95 kd transmembrane glycoprotein belonging to the immunoglobulin superfamily. CD19 is classified as a type I transmembrane protein, with a single transmembrane domain, a cytoplasmic C-terminus, and extracellular N-terminus. CD19 is expressed very early in B-cell differentiation and is only lost at terminal B-cell differentiation into plasma cells. CD19 is a biomarker for normal B cells as well as follicular dendritic cells. CD19 primarily acts as a B cell co-receptor in conjunction with CD21 and CD81. Upon activation, the cytoplasmic tail of CD19 becomes phosphorylated, which leads to binding by Src-family kinases and recruitment of PI-3 kinase.

CD19 is also expressed on all B-cell malignancies but not multiple myeloma cells. It is not expressed on other haematopoietic populations or non-haematopoietic cells and therefore targeting this antigen should not lead to toxicity to the bone marrow or non-haematopoietic organs. Loss of the normal B-cell compartment is considered an acceptable toxicity when treating lymphoid malignancies, because although effective CD19 CAR T cell therapy will result in B cell aplasia, the consequent hypogammaglobulinaemia can be treated with pooled immunoglobulin.

Different designs of CARs have been tested against CD19 in various clinical trials, as outlined in the following Table 2.

TABLE 2

| Center | Binder | Endodomain | Comment |
|---|---|---|---|
| University College | Fmc63 | CD3-Zeta | Low-level brief |
| London | | | persistence |
| Memorial Sloane | SJ25C1 | CD28-Zeta | Short-term |
| Kettering | | | persistence |
| NCI/KITE | Fmc63 | CD28-Zeta | Long-term low-level |
| | | | persistence |
| Baylor, Centre for | Fmc63 | CD3-Zeta/ | Short-term low-level |
| Cell and Gene Therapy | | CD28-Zeta | persistence |
| UPENN/Novartis | Fmc63 | 41BB-Zeta | Long-term high-level |
| | | | persistence |

As shown above, most of the studies conducted to date have used an scFv derived from the hybridoma fmc63 as part of the binding domain to recognize CD19.

The antigen-binding domain of a CAR which binds to CD19 (referred to as a CD19 CAR herein) may be any domain which is capable of binding CD19.

For example, the antigen-binding domain may comprise a CD19 antigen-binding domain as described in Table 3.

TABLE 3

| Antigen-binding | |
|---|---|
| domain | Documents |
| HD63 | Pezzutto et al., J. Immunol. Baltim. Md 1950, |
| | 138: 2793-2799 (1987) |
| 4g7 | Meeker et al., Hybridoma, 3: 305-320 (1984) |
| Fmc63 | Nicholson et al., Mol. Immunol., 34: 1157-1165 |
| | (1997) |
| B43 | Bejcek et al., Cancer Res., 55: 2346-2351 (1995) |
| SJ25C1 | Bejcek et al., supra |
| BLY3 | Bejcek et al., supra |
| B4, or | Roguska et al., Protein Eng., 9: 895-904 (1996) |
| re-surfaced, or | |
| humanized B4 | |
| HB12b, | Kansas and Tedder, Immunol. Baltim. Md 1950, 147: |
| optimized | 4094-4102 (1991); Yazawa et al., Proc. Natl. Acad. |
| and humanized | Sci. U.S.A., 102: 15178-15183 (2005); Herbst et al., |
| | J. Pharmacol. Exp. Ther., 335: 213-222 (2010) |

The gene encoding CD19 comprises ten exons: exons 1 to 4 encode the extracellular domain; exon 5 encodes the transmembrane domain; and exons 6 to 10 encode the cytoplasmic domain. The antigen-binding domain of a CD19 CAR herein may bind an epitope of CD19 encoded by exon 1 of the CD19 gene. The antigen-binding domain of a CD19 CAR herein may bind an epitope of CD19 encoded by exon 2 of the CD19 gene. The antigen-binding domain of a CD19 CAR herein may bind an epitope of CD19 encoded by exon 3 of the CD19 gene. The antigen-binding domain of a CD19 CAR herein may bind an epitope of CD19 encoded by exon 4 of the CD19 gene.

A CD19-binding domain exemplified herein comprises variable regions with complementarity determining regions (CDRs) from an antibody referred to as CAT19,
a) a heavy chain variable region (VH) having CAT19 CDRs with the following sequences:

```
  (SEQ ID NO: 37)
CDR1-GYAFSSS;
  (SEQ ID NO: 38)
CDR2-YPGDED
  (SEQ ID NO: 39)
CDR3-SLLYGDYLDY;
```

b) a light chain variable region (VL) having CAT 19 CDRs with the following sequences:

(SEQ ID NO: 40)
CDR1-SASSSVSYMH;
(SEQ ID NO: 41)
CDR2-DTSKLAS
(SEQ ID NO: 42)
CDR3-QQWNINPLT.

The CAT19 antibody is described in WO2016/139487.

It is contemplated that one or more mutations (substitutions, additions or deletions) can be introduced into one or more CDRs without negatively affecting CD19-binding activity. Each CDR may, for example, have one, two or three amino acid mutations.

The CDRs may be in the format of a single-chain variable fragment (scFv), which is a fusion protein of the heavy variable region (VH) and light chain variable region (VL) of an antibody, connected with a short linker peptide of ten to about 25 amino acids. The scFv may be in the orientation VH-VL, i.e., the VH is at the amino-terminus of the CAR molecule and the VL domain is linked to the spacer and, in turn the transmembrane domain and endodomain.

The CDRs may be grafted on to the framework of a human antibody or scFv. For example, the CAR may comprise a CD19-binding domain consisting or comprising one of the following sequences.

The CD19 CAR may comprise the following VH sequence.

VH sequence from CAT19 murine monoclonal antibody
SEQ ID NO: 43
QVQLQQSGPELVKPGASVKISCKASGYAFSSSWMNWVKQRPGKGLEWIGRI
YPGDEDTNYSGKFKDKATLTADKSSTTAYMQLSSLTSEDSAVYFCARSLLY
GDYLDYWGQGTTLTVSS

The CD19 CAR may comprise the following VL sequence.

VL sequence from CAT19 murine monoclonal antibody
SEQ ID NO: 44
QIVLTQSPAIMSASPGEKVTMTCSASSSVSYMHWYQQKSGTSPKRWIYDTS
KLASGVPDRFSGSGSGTSYFLTINNMEAEDAATYYCQQWNINPLTFGAGTK
LELKR

The CD19 CAR may comprise the following scFv sequence.

VH-VL scFv sequence from murine monoclonal antibody
SEQ ID NO: 45
QVQLQQSGPELVKPGASVKISCKASGYAFSSSWMNWVKQRPGKGLEWIGRI
YPGDEDTNYSGKFKDKATLTADKSSTTAYMQLSSLTSEDSAVYFCARSLLY
GDYLDYWGQGTTLTVSSGGGGSGGGGSGGGGSQIVLTQSPAIMSASPGEKV
TMTCSASSSVSYMHWYQQKSGTSPKRWIYDTSKLASGVPDRFSGSGSGTSY
FLTINNMEAEDAATYYCQQWNINPLTFGAGTKLELKR

The CAR may consist of or comprise one of the following sequences.

CAT19 CAR using “Campana” architecture
SEQ ID NO: 46
MGTSLLCWMALCLLGADHADAQVQLQQSGPELVKPGASVKISCKASGYAFS
SSWMNWVKQRPGKGLEWIGRIYPGDEDTNYSGKFKDKATLTADKSSTTAYM
QLSSLTSEDSAVYFCARSLLYGDYLDYWGQGTTLTVSSGGGGSGGGGSGGG
GSQIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFGAG
TKLELKRSDPTTTPAPRPPTPAPTIASQPLSLRPEACRPAAGGAVHTRGLD
FACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREEY
DVLDKRRGRDPEMGGKPRRKNPQEGLYNELQKDKMAEAYSEIGMKGERRRG
KGHDGLYQGLSTATKDTYDALHMQALPPR

“Campana” architecture refers to a CAR with a CD8a spacer and transmembrane domain, 4-1BB endodomain and TCR CD3z endodomain.

CAT19 CAR with an OX40-Zeta endodomain
SEQ ID NO: 47
MGTSLLCWMALCLLGADHADAQVQLQQSGPELVKPGASVKISCKASGYAFSSSWM
NWVKQRPGKGLEWIGRIYPGDEDTNYSGKFKDKATLTADKSSTTAYMQLSSLTSED
SAVYFCARSLLYGDYLDYWGQGTTLTVSSGGGGSGGGGSGGGGSQIVLTQSPAIM
SASPGEKVTMTCSASSSVSYMHWYQQKSGTSPKRWIYDTSKLASGVPDRFSGSGS
GTSYFLTINNMEAEDAATYYCQQWNINPLTFGAGTKLELKRSDPTTTPAPRPPTPAP
TIASQPLSLRPEACRPAAGGAVHTRGLDFACDIYIWAPLAGTCGVLLLSLVITLYCRR
DQRLPPDAHKPPGGGSFRTPIQEEQADAHSTLAKIRVKFSRSADAPAYQQGQNQLY
NELNLGRREEYDVLDKRRGRDPEMGGKPRRKNPQEGLYNELQKDKMAEAYSEIGM
KGERRRGKGHDGLYQGLSTATKDTYDALHMQALPPR
CAT19 CAR with a CD28-Zeta endodomain
SEQ ID NO: 48
MGTSLLCWMALCLLGADHADAQVQLQQSGPELVKPGASVKISCKASGYAFSSSWM
NWVKQRPGKGLEWIGRIYPGDEDTNYSGKFKDKATLTADKSSTTAYMQLSSLTSED
SAVYFCARSLLYGDYLDYWGQGTTLTVSSGGGGSGGGGSGGGGSQIVLTQSPAIM
SASPGEKVTMTCSASSSVSYMHWYQQKSGTSPKRWIYDTSKLASGVPDRFSGSGS
GTSYFLTINNMEAEDAATYYCQQWNINPLTFGAGTKLELKRSDPTTTPAPRPPTPAP
TIASQPLSLRPEACRPAAGGAVHTRGLDFACDIYIWAPLAGTCGVLLLSLVITLYCRS
KRSRLLHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
Third generation CD19 CAR
SEQ ID NO: 49
MGTSLLCWMALCLLGADHADAQVQLQQSGPELVKPGASVKISCKASGYAFSSSWM
NWVKQRPGKGLEWIGRIYPGDEDTNYSGKFKDKATLTADKSSTTAYMQLSSLTSED
SAVYFCARSLLYGDYLDYWGQGTTLTVSSGGGGSGGGGSGGGGSQIVLTQSPAIM
SASPGEKVTMTCSASSSVSYMHWYQQKSGTSPKRWIYDTSKLASGVPDRFSGSGS
GTSYFLTINNMEAEDAATYYCQQWNINPLTFGAGTKLELKRSDPTTTPAPRPPTPAP
TIASQPLSLRPEACRPAAGGAVHTRGLDFACDIFWVLVVVGGVLACYSLLVTVAFIIF
WVRSKRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELNLGRREE
YDVLDKRRGRDPEMGGKPRRKNPQEGLYNELQKDKMAEAYSEIGMKGERRRGKG
HDGLYQGLSTATKDTYDALHMQALPPR
CD19 CAR with IgG1 hinge spacer
SEQ ID NO: 50
MGTSLLCWMALCLLGADHADAQVQLQQSGPELVKPGASVKISCKASGYAFSSSWM
NWVKQRPGKGLEWIGRIYPGDEDTNYSGKFKDKATLTADKSSTTAYMQLSSLTSED
SAVYFCARSLLYGDYLDYWGQGTTLTVSSGGGGSGGGGSGGGGSQIVLTQSPAIM
SASPGEKVTMTCSASSSVSYMHWYQQKSGTSPKRWIYDTSKLASGVPDRFSGSGS
GTSYFLTINNMEAEDAATYYCQQWNINPLTFGAGTKLELKRSDPAEPKSPDKTHTCP
PCPKDPKFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPPR
CD19 CAR with hinge-CH2-CH3 of human IgG1 with FcR binding
sites mutated out
SEQ ID NO: 51
MGTSLLCWMALCLLGADHADAQVQLQQSGPELVKPGASVKISCKASGYAFSSSWM
NWVKQRPGKGLEWIGRIYPGDEDTNYSGKFKDKATLTADKSSTTAYMQLSSLTSED
SAVYFCARSLLYGDYLDYWGQGTTLTVSSGGGGSGGGGSGGGGSQIVLTQSPAIM
SASPGEKVTMTCSASSSVSYMHWYQQKSGTSPKRWIYDTSKLASGVPDRFSGSGS
GTSYFLTINNMEAEDAATYYCQQWNINPLTFGAGTKLELKRSDPAEPKSPDKTHTCP
PCPAPPVAGPSVFLFPPKPKDTLMIARTPEVTCVVVDVSHEDPEVKFNWYVDGVEV
HNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGKKDPKF
WVLVVVGGVLACYSLLVTVAFIIFWVRSKRSRLLHSDYMNMTPRRPGPTRKHYQPY
APPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATK
DTYDALHMQALPPR

The CAR provided herein may comprise a variant of the polypeptide of SEQ ID NO: 37-51 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at http-//blast.ncbi.nlm.nih.gov.

The CD19 CAR exemplified herein (i.e., the CAT19CAR using “Campana” architecture, SEQ ID NO: 46) has properties contemplated by the disclosure to result in lower toxicity and better efficacy in treated patients. When compared with an fmc63-Campana CAR, the CAT19CAR exemplified herein effected killing of target cells expressing CD19 and proliferated in response to CD19 expressing targets, but Interferon-gamma release was less. Further, a small animal model of an aggressive B-cell lymphoma showed equal efficacy and equal engraftment between the fmc63- and CAT19-based CAR-T cells, but surprisingly, less of the CAT19 CAR T-cells were exhausted than fmc63 CAR T-cells. See, Examples 2 and 3 of US Publication No.: 2018-0044417.

The CAT19CAR provided herein may cause 25, 50, 70 or 90% lower IFNγ release in a comparative assay involving bringing CAR T cells into contact with target cells.

The CAT19CAR provided herein may result in a smaller proportion of CAR T cells becoming exhausted than fmc63 CAR T cells. T cell exhaustion may be assessed using methods known in the art, such as analysis of PD-1 expression. The CAR may cause 20, 30, 40, 50, 60 of 70% fewer CAR T cells to express PD-1 that fmc63 CAR T cells in a comparative assay involving bringing CAR T cells into contact with target cells.

Another exemplary CD19 antigen-binding domain contemplated by the disclosure is based on the CD19 antigen-binding domain CD19ALAb (described in WO2016/102965) and comprises:
a) a heavy chain variable region (VH) having CDRs with the following sequences:

(SEQ ID NO: 52)
CDR1-SYWMN;
(SEQ ID NO: 53)
CDR2-QIWPGDGDTNYNGKFK
(SEQ ID NO: 54)
CDR3-RETTTVGRYYYAMDY;

b) a light chain variable region (VL) having CDRs with the following sequences:

```
  (SEQ ID NO: 55)
CDR1-KASQSVDYDGDSYLN;
  (SEQ ID NO: 56)
CDR2-DASNLVS
(SEQ ID NO: 57)
CDR3-QQSTEDPWT.
```

It is contemplated that it is possible to introduce one or more mutations (substitutions, additions or deletions) into one or more CDRs without negatively affecting CD19-binding activity. Each CDR may, for example, have one, two or three amino acid mutations.

The CAR may comprise one of the following amino acid sequences.

Murine CD19ALAb scFv sequence
SEQ ID NO: 58
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSSDIQLTQSPASLAVSLGQRATISC
KASQSVDYDGDSYLNWYQQIPGQPPKLLIYDASNLVSGIPPRFSGSGSG
TDFTLNIHPVEKVDAATYHCQQSTEDPWTFGGGTKLEIK
Humanized CD19ALAb scFv sequenceHeavy 19, Kappa 16
SEQ ID NO: 59
QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSSDIQLTQSPDSLAVSLGERATINC
KASQSVDYDGDSYLNWYQQKPGQPPKLLIYDASNLVSGVPDRFSGSGSG
TDFTLTISSLQAADVAVYHCQQSTEDPWTFGQGTKVEIKR
(Humanized CD19ALAb scFv sequenceHeavy 19,
Kappa 7)
SEQ ID NO: 60
QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSSDIQLTQSPDSLAVSLGERATINC
KASQSVDYDGDSYLNWYQQKPGQPPKVLIYDASNLVSGVPDRFSGSGSG
TDFTLTISSLQAADVAVYYCQQSTEDPWTFGQGTKVEIKR

The scFv may be in a VH-VL orientation (as shown in SEQ ID NO:s 45, 58-60) or a VL-VH orientation.

The CAR may comprise one of the following VH sequences:

Murine CD19ALAb VH sequence
SEQ ID NO: 61
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSS
Humanized CD19ALAb VH sequence
SEQ ID NO: 62
QVQLVQSGAEVKKPGASVKLSCKASGYAFSSYWMNWVRQAPGQSLEWIGQ
IWPGDGDTNYNGKFKGRATLTADESARTAYMELSSLRSGDTAVYFCARRE
TTTVGRYYYAMDYWGKGTLVTVSS

The CAR may comprise one of the following VL sequences:

Murine CD19ALAb VL sequence
SEQ ID NO: 63
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK
(Humanized CD19ALAb VL sequence, Kappa 16)
SEQ ID NO: 64
DIQLTQSPDSLAVSLGERATINCKASQSVDYDGDSYLNWYQQKPGQPPKL
LIYDASNLVSGVPDRFSGSGSGTDFTLTISSLQAADVAVYHCQQSTEDPW
TFGQGTKVEIKR
Humanized CD19ALAb VL sequence, Kappa 7
SEQ ID NO: 65
DIQLTQSPDSLAVSLGERATINCKASQSVDYDGDSYLNWYQQKPGQPPKV
LIYDASNLVSGVPDRFSGSGSGTDFTLTISSLQAADVAVYYCQQSTEDPW
TFGQGTKVEIKR

The CAR provided herein may comprise a variant of the sequence shown as any of SEQ ID NO: 52-65 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at blast.ncbi.nlm.nih.gov.

### Signal Peptides

The CARs of the cell may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the amino acid sequence of any of SEQ ID NO: 68-70 or a variant thereof having 5, 4, 3, 2 or 1 amino acid mutations (insertions, substitutions or additions) provided that the signal peptide still functions to cause cell surface expression of the CAR.

The signal peptide of SEQ ID NO: 66 is compact and highly efficient. It is predicted to give about 95% cleavage after the terminal glycine, giving efficient removal by signal peptidase.

SEQ ID NO: 66
MGTSLLCWMALCLLGADHADA

The signal peptide of SEQ ID NO: 67 follows.

METDTLLLWVLLLLVPGSTG

The signal peptide of SEQ ID NO: 68 follows.

METDTLILWVLLLLVPGSTG

The signal peptide of SEQ ID NO: 69 follows.

MGWSCIILFLVATATGVHS

The signal peptide of SEQ ID NO: 70 is derived from IgG1.

SEQ ID NO: 70:
MSLPVTALLLPLALLLHAARP

The signal peptide of SEQ ID NO: 71 is derived from CD8.

SEQ ID NO: 71:
MAVPTQVLGLLLLWLTDARC

The signal peptide for the first CAR may have a different sequence from the signal peptide of the second CAR.

### Spacers

CARs comprise a spacer to connect the antigen-binding domain with the transmembrane domain and spatially separate the antigen-binding domain from the endodomain. A flexible spacer allows the antigen-binding domain to orient in different directions to facilitate binding.

The spacer may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

In the cells provided herein, the first and second CARs may comprise different spacer molecules. For example, the spacer may, for example, comprise an IgG1 Fc region, an IgG1 hinge or a human CD8 stalk or the mouse CD8 stalk. The spacer may alternatively comprise an alternative linker which has similar length and/or domain spacing properties as an IgG1 Fc region, an IgG1 hinge or a CD8 stalk. A human IgG1 spacer may be altered to remove Fc binding motifs.

The spacer for the CD19 CAR may comprise a CD8 stalk spacer, or a spacer having a length equivalent to a CD8 stalk spacer. The spacer for the CD19 CAR may have at least 30 amino acids or at least 40 amino acids. It may have between 35-55 amino acids, for example between 40-50 amino acids. It may have about 46 amino acids.

The spacer for the BCMA CAR may comprise an IgG1 hinge spacer, or a spacer having a length equivalent to an IgG1 hinge spacer. The spacer for the BCMA CAR may have fewer than 30 amino acids or fewer than 25 amino acids. It may have between 15-25 amino acids, for example between 18-22 amino acids. It may have about 20 amino acids.

Examples of amino acid sequences for these spacers are given below:

(hinge-CH2CH3 of human IgG1)
SEQ ID NO: 72
AEPKSPDKTHTCPPCPAPPVAGPSVFLFPPKPKDTLMIARTPEVTCVVVDVSHEDPE
VKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQK
SLSLSPGKKD
(human CD8 stalk):
SEQ ID NO: 73
TTTPAPRPPTPAPTIASQPLSLRPEACRPAAGGAVHTRGLDFACD
(human IgG1 hinge):
SEQ ID NO: 74
AEPKSPDKTHTCPPCPKDPK
(human IgG1 hinge variation)
SEQ ID NO: 75
EPKSCDKTHTCPPCP
(IgG1 Hinge-Fc)
SEQ ID NO: 76
AEPKSPDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQK
SLSLSPGKKDPK
(IgG1 Hinge-Fc modified to remove Fc receptor recognition motifs)
SEQ ID NO: 77
AEPKSPDKTHTCPPCPAPPVA*GPSVFLFPPKPKDTLMIARTPEVTCVVVDVSHEDP
EVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQK
SLSLSPGKKDPK
Modified residues are underlined; * denotes a deletion.
(CD2 ectodomain)
SEQ ID NO: 78
KEITNALETWGALGQDINLDIPSFQMSDDIDDIKWEKTSDKKKIAQFRKEKETFKEKD
TYKLFKNGTLKIKHLKTDDQDIYKVSIYDTKGKNVLEKIFDLKIQERVSKPKISWTCINT
TLTCEVMNGTDPELNLYQDGKHLKLSQRVITHKWTTSLSAKFKCTAGNKVSKESSV
EPVSCPEKGLD
(CD34 ectodomain)
SEQ ID NO: 79
SLDNNGTATPELPTQGTFSNVSTNVSYQETTTPSTLGSTSLHPVSQHGNEATTNITE
TTVKFTSTSVITSVYGNTNSSVQSQTSVISTVFTTPANVSTPETTLKPSLSPGNVSDL
STTSTSLATSPTKPYTSSSPILSDIKAEIKCSGIREVKLTQGICLEQNKTSSCAEFKKD
RGEGLARVLCGEEQADADAGAQVCSLLLAQSEVRPQCLLLVLANRTEISSKLQLMK
KHQSDLKKLGILDFTEQDVASHQSYSQKT

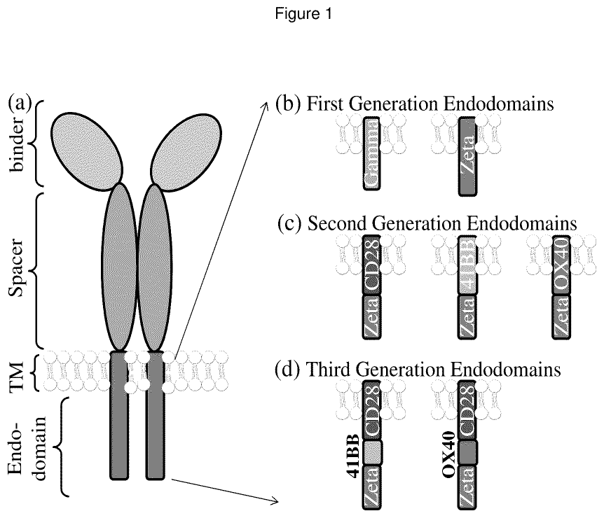

Since CARs are typically homodimers (see FIG. 1A), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. The spacer of the first CAR may be sufficiently different from the spacer of the second CAR in order to avoid cross-pairing. The amino acid sequence of the first spacer may share less than 50%, 40%, 30% or 20% identity at the amino acid level with the second spacer.

### Transmembrane Domains

The transmembrane domain is the domain of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion provided herein. The presence and span of a transmembrane domain of a protein can be determined by those skilled in the art using the TMHMM algorithm (http-//www.cbs.dtu.dk/services/TMHMM-2.0/). Further, given that the transmembrane domain of a protein is a relatively simple structure, i.e, a polypeptide predicted to form a hydrophobic alpha helix of sufficient length to span the membrane, an artificially designed transmembrane domain may also be used (U.S. Pat. No. 7,052,906 B1 describes synthetic transmembrane components).

The transmembrane domain may be derived from CD28, which gives good receptor stability. The CD28 transmembrane domain sequence is shown as SEQ ID NO: 80

SEQ ID NO: 80 FWVLVVVGGVLACYSLLVTVAFIIFWV

The transmembrane domain may be derived from human Tyrp-1. The tyrp-1 transmembrane domain sequence is shown as SEQ ID NO: 81.

SEQ ID NO: 81 IIAIAVVGALLLVALIFGTASYLI

The transmembrane domain may be derived from CD8A. The CD8A transmembrane domain sequence is shown as SEQ ID NO: 82.

SEQ ID NO: 82 IYIWAPLAGTCGVLLLSLVITLYC

### Endodomains

As noted above, the endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains three ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The cells provided herein comprise two CARs, each with an endodomain.

The endodomain of the first CAR and the endodomain of the second CAR may comprise: (i) an ITAM-containing endodomain, such as the endodomain from CD3 zeta; and/or (ii) a co-stimulatory domain, such as the endodomain from CD28; and/or (iii) a domain which transmits a survival signal, for example a TNF receptor family endodomain such as OX-40 or 4-1BB.

Thus, the endodomain of the CAR of the present disclosure may comprise combinations of one or more of the CD3-Zeta endodomain, the 41BB endodomain, the OX40 endodomain or the CD28 endodomain.

The intracellular T-cell signalling domain (endodomain) of the CAR of the present disclosure may comprise the sequence shown as any of SEQ ID NO: 83-90 or a variant thereof having at least 80% sequence identity.

(CD3 zeta endodomain)
SEQ ID NO: 83
RSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTA
TKDTYDALHMQALPPR
(41BB endodomain)
SEQ ID NO: 84
KRGRKKLLYIFKQPFMRPVQTTQEEDGCSCRFPEEEEGGCEL
(OX40 endodomain)
SEQ ID NO: 85
RRDQRLPPDAHKPPGGGSFRTPIQEEQADAHSTLAKI
(CD28 endodomain)
SEQ ID NO: 86
KRSRLLHSDYMNMTPRRPGPTRKHYQPYAPPRDFAAY

Examples of combinations of such endodomains include 41 BB-Zeta, OX40-Zeta, CD28-Zeta and CD28-OX40-Zeta.

(41BB-Zeta endodomain fusion)
SEQ ID NO: 87
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR
(OX40-Zeta endodomain fusion)
SEQ ID NO: 88
RRDQRLPPDAHKPPGGGSFRTPIQEEQADAHSTLAKIRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
(CD28Zeta endodomain fusion)
SEQ ID NO: 89
KRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
(CD28OXZeta)
SEQ ID NO: 90
KRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

A variant sequence may have at least 80%, 85%, 90%, 95%, 98% or 99% sequence identity to any of SEQ ID NO: 83-90 provided that the sequence provides an effective transmembrane domain/intracellular T cell signaling domain.

### Nucleic Acids

One or more nucleic acid(s) provided herein encode a BCMA CAR and a CD19 CAR of the disclosure. As used herein, the terms “polynucleotide”, “nucleotide”, and “nucleic acid” are intended to be synonymous with each other.

The nucleic acid may be, for example, an RNA, a DNA or a cDNA. Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

Alternative codons may be used in regions of sequence encoding the same or similar amino acid sequences, in order to avoid homologous recombination when the both CARs are encoded by the same vector.

Due to the degeneracy of the genetic code, it is possible to use alternative codons which encode the same amino acid sequence. For example, the codons “ccg” and “cca” both encode the amino acid proline, so using “ccg” may be exchanged for “cca” without affecting the amino acid in this position in the sequence of the translated protein.

The alternative RNA codons which may be used to encode each amino acid are summarized in Table 4.

TABLE 4

| | U | | | C | | | A | | | G | | |
|---|---|---|---|---|---|---|---|---|---|---|---|---|
| U | UUU | —— | | UCU | —— | | UAU | —— | | UGU | —— | |
| | | {close oversize brace} | Phe (F) | UCC | | | | {close oversize brace} | Tyr (Y) | | {close oversize brace} | Cys (C) |
| | UUC | | | UCA | {close oversize brace} | Ser (S) | UAC | | | UGC | | |
| | UUA | | | UCG | | | UAA | | Ocher | UGA | | Opal |
| | | {close oversize brace} | Leu (L) | | | | UAG | | Amber | | {close oversize brace} | |
| C | UUG | | | | | | | | | UGG | | Trp(W) |
| | CUU | | | CCU | | | CAU | | | CGU | | |
| | CUC | | | CCC | | | | {close oversize brace} | His (H) | CGC | | |
| | CUA | {close oversize brace} | Leu (L) | CCA | {close oversize brace} | Pro (P) | CAC | | | CGA | {close oversize brace} | Arg (R) |
| | CUG | | | CCG | | | CAA | | | CGG | | |
| | | | | | | | | {close oversize brace} | Gln (Q) | | | |
| | | | | | | | CAG | | | | | |
| A | AUU | | | ACU | | | AAU | | | AGU | | |
| | AUC | {close oversize brace} | Ile (I) | ACC | | | | {close oversize brace} | Asn (N) | | {close oversize brace} | Ser (S) |
| | AUA | | | ACG | {close oversize brace} | Thr (T) | AAC | | | AGC | | |
| | AUG | | Met(M) | ACG | | | AAA | | | AGA | | |
| | | | | | | | | {close oversize brace} | Lys (K) | | {close oversize brace} | Arg (R) |
| | | | | | | | AAG | | | AGG | | |
| G | GUU | | | GCU | | | GAU | | | GGU | | |
| | GUC | | | GCC | | | | {close oversize brace} | Asp (D) | GGC | | |
| | GUA | {close oversize brace} | Val (V) | GCA | {close oversize brace} | Ala (A) | GAU | | | GGA | {close oversize brace} | Gly (G) |
| | GUG | | | GCG | | | GAA | | | GGG | | |
| | | | | | | | | {close oversize brace} | Glu (E) | | | |
| | | | | | | | GAG | | | | | |

Alternative codons may be used in the portions of nucleic acid which encode the spacer of the first CAR and the spacer of the second CAR, especially if the same or similar spacers are used in the first and second CARs.

Alternative codons may be used in the portions of nucleic acid which encode the transmembrane domain of the first CAR and the transmembrane of the second CAR, especially if the same or similar transmembrane domains are used in the first and second CARs.

Alternative codons may be used in one or more nucleic acids which encode co-stimulatory domains, such as the CD28 endodomain.

Alternative codons may be used in one or more domains which transmit survival signals, such as OX40 and 41BB endodomains.

Alternative codons may be used in the portions of nucleic acid encoding a CD3zeta endodomain and/or the portions of nucleic acid encoding one or more costimulatory domain(s) and/or the portions of nucleic acid encoding one or more domain(s) which transmit survival signals.

Nucleic Acid Construct The present disclosure also provides a nucleic acid construct encoding a chimeric receptor of the disclosure.

A nucleic acid construct encoding a FabCAR (FIG. 2a) may have the structure:
VH-CH-spacer-TM-endo-coexpr-VL-CL or
VL-CL-spacer-TM-endo-coexpr-VH-CH
in which:
VH is a nucleic acid sequence encoding a heavy chain variable region;
CH is a nucleic acid sequence encoding a heavy chain constant region spacer is a nucleic acid encoding a spacer;
TM is a nucleic acid sequence encoding a transmembrane domain;
endo is a nucleic acid sequence encoding an endodomain;
coexpr is a nucleic acid sequence enabling co-expression of the first and second polypeptides;
VL is a nucleic acid sequence encoding a light chain variable region; and
CL is a nucleic acid sequence encoding a light chain constant region.

For both structures mentioned above, nucleic acid sequences encoding the two polypeptides may be in either order in the construct.

There is also provided a nucleic acid construct encoding an OR gate, which comprises two of more CARs, at least one of which is a FabCAR according to the present disclosure.

A nucleic acid construct encoding a double OR gate may have the structure:
VH-CH-spacer1-TM1-endo1-coexpr1-VL-CL-coexpr2-AgBD-spacer2-TM2-endo2; or
VL-CL-spacer-TM1-endo1-coexpr1-VH-CH-coexpr2-AgBD-spacer2-TM2-endo2
in which:
VH is a nucleic acid sequence encoding a heavy chain variable region of the first CAR;
CH is a nucleic acid sequence encoding a heavy chain constant region of the first CAR; Spacer 1 is a nucleic acid sequence encoding a spacer of the first CAR; TM1 is a nucleic acid sequence encoding a transmembrane domain of the first CAR;
Endo1 is a nucleic acid sequence encoding an endodomain of the first CAR;
Coexpr1 and coexpr2, which my be the same or different, are nucleic acid sequences enabling co-expression of the first and second polypeptides of the first CAR; and the first and second CARs;
VL is a nucleic acid sequence encoding a light chain variable region of the first CAR;
CL is a nucleic acid sequence encoding a light chain constant region of the first CAR;
AgBD is a nucleic acid sequence encoding an antigen binding domain of the second CAR;
Spacer2 is a nucleic acid sequence encoding a spacer of the second CAR;
TM2 is a nucleic acid sequence encoding a transmembrane domain of the second CAR; and
Endo2 is a nucleic acid sequence encoding an endodomain of the second CAR.

The antigen-binding domain of the second CAR (AgBD) may, for example, be an scFv or a domain antbody or single domain antibody dAb.

For both structures mentioned above, nucleic acid sequences encoding the two polypeptides of the first CAR; and the nucleic acid sequences encoding the first and second CARs may be in any order in the construct.

A nucleic acid construct encoding a double FabCAR OR gate may have the structure:
VH1-CH1-spacer1-TM1-endo1-coexpr1-VL1-CL1-coexpr2-VH2-CH2-spacer2-TM2-endo2-coexpr3-VL2-CL2;
VH1-CH1-spacer1-TM1-endo1-coexpr1-VL1-CL1-coexpr2-VL2-CL2-spacer2-TM2-endo2-coexpr3-VH2-CH2;
VL1-CL1-spacer1-TM1-endo1-coexpr1-VH1-CH1-coexpr2-VL2-CL2-spacer2-TM2-endo2-coexpr3-VH2-CH2; or
VL1-CL1-spacer1-TM1-endo1-coexpr1-VH1-CH1-coexpr2-VH2-CH2-spacer2-TM2-endo2-coexpr3-VL2-CL2;
in which:
VH1 is a nucleic acid sequence encoding a heavy chain variable region of the first CAR;
CH1 is a nucleic acid sequence encoding a heavy chain constant region of the first CAR;
Spacer 1 is a nucleic acid sequence encoding a spacer of the first CAR;
TM1 is a nucleic acid sequence encoding a transmembrane domain of the first CAR;
Endo1 is a nucleic acid sequence encoding an endodomain of the first CAR;
Coexpr1, coexpr2, and coexpr 3 which may be the same or different, are nucleic acid sequences enabling co-expression of the first and second polypeptides of the first CAR; and the first and second polypeptides of the second CAR;
VL2 is a nucleic acid sequence encoding a light chain variable region of the second CAR;
CL2 is a nucleic acid sequence encoding a light chain constant region of the second CAR;
VH2 is a nucleic acid sequence encoding a heavy chain variable region of the second CAR;
CH2 is a nucleic acid sequence encoding a heavy chain constant region of the second CAR;
Spacer 2 is a nucleic acid sequence encoding a spacer of the second CAR; TM2 is a nucleic acid sequence encoding a transmembrane domain of the second CAR;
Endo2 is a nucleic acid sequence encoding an endodomain of the second CAR;
VL2 is a nucleic acid sequence encoding a light chain variable region of the second CAR;
CL2 is a nucleic acid sequence encoding a light chain constant region of the second CAR.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the disclosure may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present disclosure, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picomaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID NO: 91 (RAEGRGSLLTCGDVEENPGP) or SEQ ID NO: 92 (ATNFSLLKQAGDVEENPGP).

### Vectors

The present disclosure also provides a vector, or kit of vectors which comprises one or more CAR-encoding nucleic acid(s). Such a vector may be used to introduce the nucleic acid(s) into a host cell so that it expresses the first and second CARs.

The vector may, for example, be a plasmid or a viral vector, such as a retroviral vector or a lentiviral vector, or a transposon-based vector or synthetic mRNA.

The vector may be capable of transfecting or transducing a T cell.

### Cells

Cells are provided herein which co-express a first CAR and a second CAR, wherein one CAR binds BCMA and the other CAR binds CD19, such that the cell recognizes a target cell expressing either of these markers. Populations of cells which comprise cells which co-express a BCMA CAR and a CD19 CAR, as well as cells that express the BCMA CAR and cells that express the CD19 CAR are also provided.

Double transduction has several advantages.
1. Two separate products would not allow “piggybacking” of BCMA specificity onto long persisting CD19 CAR T cells. CAT19 CAR T-cell persistence is well demonstrated. Reported BCMA CAR T-cell persistence is typically short-lived. This may be due to intrinsic properties of BCMA CARs. Alternatively, this may also be due to reduced signaling due to lower BCMA target density or other factors. Studying the long-term engraftment of single/double-positive populations will help elucidate this. For instance, long-term engraftment of only single-positive CD19 CAR T-cells suggests an intrinsic effect of a CAR; long-term engraftment of only CD19 CAR T-cells (both single and double) would suggest that higher antigen targeting is needed for persistence.
2. Effects of different expression or stoichiometry can be studied. If different relative expression of BCMA vs CD19 CAR are required for optimal persistence, an optimal ratio of expression or co-expression can be elucidated by measuring the CAR expression on long-term engrafted cells.
3. Immune response against the transgene products may be reduced. If two potentially immunogenic binders are encoded in the same expression cassette, the probability of triggering and immune response doubles. With double transduction the probability that at least one population will persist increases.

The cell may be any eukaryotic cell capable of expressing a CAR at the cell surface, such as an immunological cell.

In particular, the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarized below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The T cell provided herein may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR-expressing cells provided herein may be any of the cell types mentioned above.

CAR-expressing cells, such as CAR-expressing T or NK cells may either be created ex vivo either from a patient's own peripheral blood (1a party), or in the setting of a haematopoietic stem cell transplant from donor peripheral blood (2^{nd} party), or peripheral blood from an unconnected donor (3^{rd} party).

The present disclosure also provides a cell composition comprising CAR-expressing T cells and/or CAR-expressing NK cells, which cells co-express a CAR that binds CD19 and another CAR that binds CD22, such that the cells can recognize a target cell expressing either of these markers. The cell composition may be made by transducing a blood-sample ex vivo with a nucleic acid according to the present disclosure.

The term “CD19/22 CAR T-cells” refers herein to a cell composition comprising a mixture of untransduced cells, cells expressing a CD19 CAR alone, cells expressing a CD22 CAR alone, and cells expressing both the CD19 and CD22 CARs.

Alternatively, T or NK cells provided herein may be derived from ex vivo differentiation of inducible progenitor cells or embryonic progenitor cells to T or NK cells.

Alternatively, an immortalized T-cell line which retains its lytic function and could act as a therapeutic may be used.

The CAR cells are generated by introducing DNA or RNA coding for the CARs by one of many means including, but not limited to, transduction with a viral vector, transfection with DNA or RNA. Cells may be activated and/or expanded prior to being transduced with CAR-encoding nucleic acid, for example by treatment with an anti-CD3 monoclonal antibody.

The T or NK cells provided herein may be made by: (i) isolation of a T or NK cell-containing sample from a subject or other sources listed above, and (ii) transduction or transfection of the T or NK cells with one or more a nucleic acid(s) encoding the CD19 and CD22 CARs.

The T or NK cells may then by purified, for example, selected on the basis of expression of the antigen-binding domain of the antigen-binding polypeptide.

Pharmaceutical compositions The present disclosure also relates to a pharmaceutical composition containing a plurality of CAR-expressing cells, such as T cells or NK cells provided herein.

Pharmaceutical compositions comprising the BCMA/CD19 CAR T-cell product described in Example 3 are provided. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Methods of Treatment

The cell compositions of the present disclosure, for example the BCMA/CD19 CAR T-cell product composition described in Example 3, are capable of killing cancer cells recognizable by expression of BCMA or CD19, such as multiple myeloma (MM) cells.

CAR-expressing cells, such as T cells, may either be created ex vivo either from a patient's own peripheral blood (1a party), or in the setting of a haematopoietic stem cell transplant from donor peripheral blood (2^{rd} party), or peripheral blood from an unconnected donor (3^{rd} party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

Examples of malignancies which express BCMA CD19 or CD22 are multiple myeloma (MM) or plasma cell disorders such as plasmacytoma, plasma cell leukemia, multiple myeloma, macroglobulinemia,amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The cell compositions of the present disclosure may be used in the treatment of MM. MM may be relapsed and/or refractory MM.

Treatment with the T cells provided herein is contemplated to help prevent the escape or release of tumor cells which often occurs with standard care approaches.

The methods provided herein slow or prevent progression of the cancer, diminish the extent of the cancer, result in remission (partial or total) of the cancer, and/or prolong survival of the patient.

In the provided methods, the patient treated has a relapsed or resistant BCMA+ or CD19+ malignancy.

Where the relapsed or resistant BCMA+ or CD19+ malignancy is MM, there are several parameters that may be used to define relapsed or resistant MM:
a) relapsed or resistant MM
b) Secretory disease;
c) ≥3 prior lines of therapy;
d) Refractory to last line of therapy;
e) Has previously received or is not suitable for ASCT.

Secretory disease is defined as PP a 5 g/L and/or sFLC a 100 mg/L of involved light chain with abnormal K:L ratio.

A MM patient that has had 3 or more prior lines of therapy may be a patient who has received therapy including proteasome inhibitor, ImiD, and anti CD38 antibody A patient having MM that is refractory to last line of therapy may be a patient who has not achieved at least partial response (PR) and progressed within 60 days of last dose, or achieved at least PR but progressed within 6 months of last dose of therapy.

The patient may be administered a single dose of 5×10⁶ CAR T-cells, such as BCMA/CD19 CAR T-cell product described in Example 3. The patient may be administered a single dose of 50×10⁶ CAR T-cells, such as BCMA/CD19 CAR T-cell product described in Example 3. The patient may be administered a single dose of 150×10⁶ CAR T-cells, such as the BCMA/CD19 CAR T-cell product described in Example 3. The patient may be administered a single dose of 300×10⁶ CAR T-cells, such as the BCMA/CD19 CAR T-cell product described in Example 3. The patient may be administered a single dose of 25×10⁶ CAR T-cells, such as the BCMA/CD19 CAR T-cell product described in Example 3. The administration may be an intravenous injection through a Hickman line or peripherally inserted central catheter (PICC line).

The patient may be administered conditioning chemotherapy or lymphodepletion prior to receiving the CAR T-cells. The conditioning chemotherapy or lymphodepletion may include cyclophosphamide and fludarabine, such as 300 mg/m² cyclophosphamide×3 doses on Day −5 and Day −3 and 30 mg/m2 fludarabine for 3 doses over Day −5 to Day −3 prior to BCMA/CD19 CAR T-cell product infusion on Day 0.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

### Example 1

### Discovery and Selection of the D8 Binder

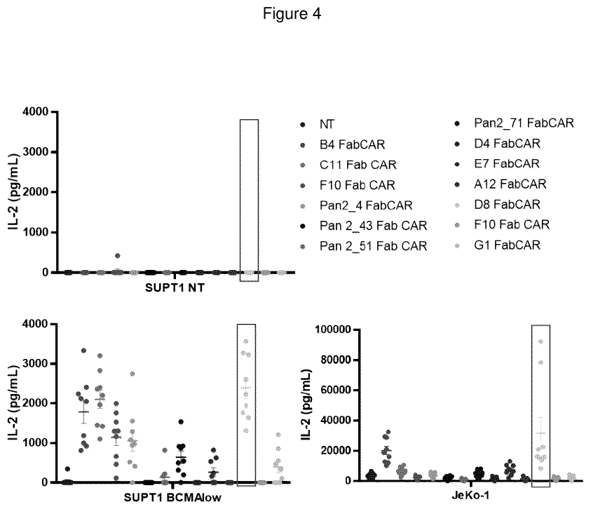

A key desired feature of a BCMA CAR was sensitivity to low density antigen. Hence, a set of BCMA binders were generated by immunizing rats with human BCMA and sequencing subsequent hybridomas. Derived binder variable heavy chain (VH) and variable light chain (VL) sequences were cloned into a CAR format and functionally tested in primary human T cells against a variety of targets including targets which express BCMA at very low density. Since a proportion of VHNL's isolated from hybridomas do not fold well as scFv, screening was performed in Fab CAR format (FIG. 4).

Target cells include the T cell lymphoma-derived cell line SupT1, which does not naturally express BCMA. This was used as a negative control cell line. SupT1 cells were engineered in-house to express low levels of BCMA (SupT1.BCMAlow), measured at approximately 600 copies/cell. The JeKo-1 cell line, a mantle cell lymphoma derived cell line which expresses very low levels of BCMA at around 100 copies/cell was also used in this study. For information, MM.1s, which is a myeloma cell line, and which expresses approximately 4000 BCMA copies/cell, and SupT1 cells engineered in-house to have approximately 10,000 BCMA copies/cell (SupT1.BCMAhigh) were used in subsequent experiments.

There is a hierarchy of T cell activation, and IL-2 secretion denotes full activation. Consequently, binder and CAR D8 which triggered more IL-2 secretion in response to a low-density target was selected for further characterization.

### Binding Characteristics of D8

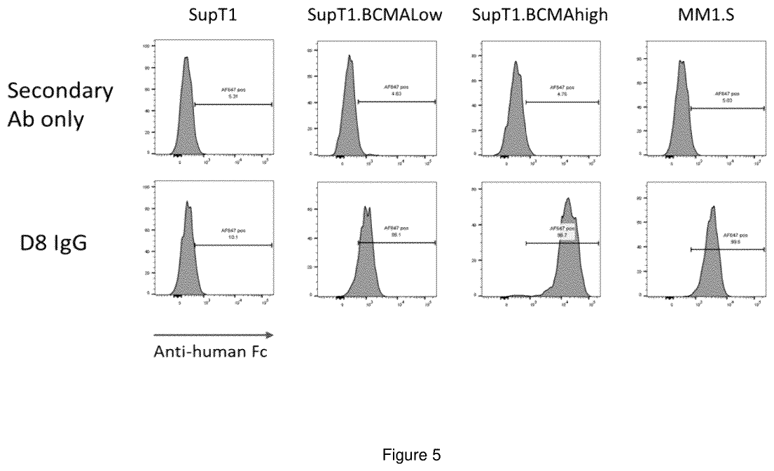

To study binding of D8 to BCMA, the D8 VH and VL domains were cloned into an IgG format and produced as a recombinant protein from expiCHO cells. To determine D8 IgG binding to BCMA on cell surface, SupT1 cells, SupT1.BCMAhigh, SupT1.BCMAlow and MM1.s cells were stained with D8 IgG and a fluorescent secondary antibody and binding determined by flow-cytometry (FIG. 5). Selective binding of D8 IgG to MM1.s and both SupT1.BCMAhigh and SupT1.BCMAlo compared with SupT1 cells was demonstrated.

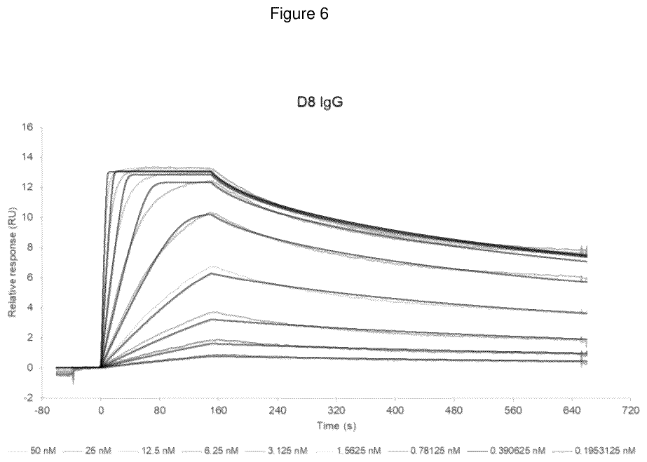

D8 IgG binding to soluble BCMA ectodomain was also studied using surface plasmon resonance (FIG. 6). Kinetic affinity (KD) was measured at 0.126 nM for D8 IgG.

Tissue Cross-Reactivity Studies with D8

Tissue cross-reactivity studies were outsourced to Citoxlab France.

GLP Tissue Cross Reactivity Study: Citoxlab France, study No. 47714: D8 IgG was tested in a GLP tissue cross reactivity study using semi-automated method on a Ventana Discovery XT platform, at two concentrations of 1.25 and 5 μg/mL, on a panel of 42 frozen human tissues and blood smears. D8 IgG produced minimal staining of scattered lymphoid-type cells in the small intestine (duodenum, jejunum) and in the prostate (stroma). Cell morphology and tissue distribution were consistent with the expected profile of BCMA expression in plasma cells.

It was concluded that D8 had no cross-reactive binding in a tissue cross-reactivity study.

### Selection of Fab Format for D8 CAR

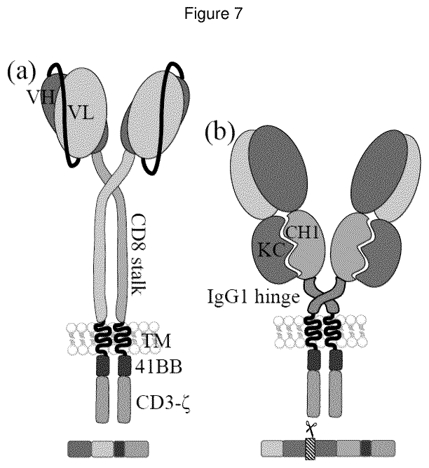

Following selection of D8 as a binder which confers high BCMA sensitivity in a CAR format and which is specific for BCMA, the CAR architecture for optimal function was determined. Screening was performed in FabCAR format since a proportion of binders do not work well as scFv. A standard D8 CAR in a traditional Campana format using a scFv as binding domain was generated (FIG. 7).

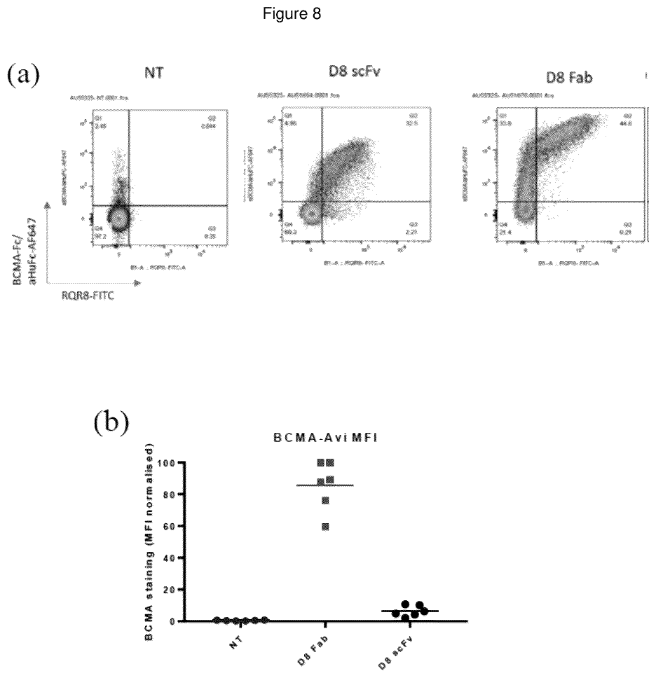

Firstly, expression of D8Fab-41BBζ compared with D8-41BBζ was tested. Normal donor T cells were transduced with vectors expressing each. For these experiments, the surface marker gene RQR8(130) was co-expressed to control for transduction. Staining with recombinant biotinylated BCMA allowed determination of CAR stability. The median fluorescence intensity (MFI) for BCMA-Fc staining (used as an indicator of CAR density on cell surface) was considerably higher for D8 in Fab format compared to scFv; where the mean MFI values were 85.46±15.46 or 6.415±3.427 AU for D8 Fab or scFv respectively (FIG. 8).

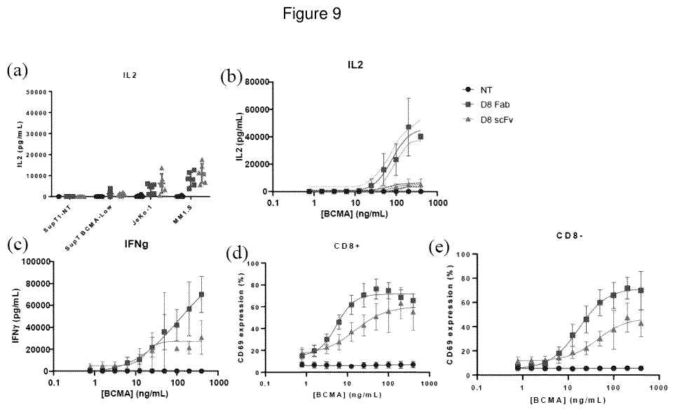

Next, CAR functionality of BCMA-CAR with D8 binder in either scFv or Fab format were compared in vitro, by measuring cytokine production in co-cultures with BCMA-expressing targets. There was a trend to lower IL-2 production by D8-41BB-ζ vs D8Fab-41BB-ζ expressing T cells in response to JeKo-1 target cells (FIG. 9(a)). To determine sensitivity more finely, CAR T cells were tested against decreasing density of plate-bound antigen. CAR T cells were cultured on plates which had been incubated with dilutions of recombinant BCMA-Fc. D8Fab-41BB-ζ CAR T cells showed superior IFN-gamma and IL-2 release. Further, CD69, a marker of activation on of CD69 was also increased in Fab vs scFv format.

### In Vitro Performance of D8-Fab-41BBC CAR

The function of D8Fab-41BBζ transduced T cells against BCMA-expressing targets was next evaluated in vitro. To match the final manufacturing process, D8 CAR T cells were generated in a research setting on a small scale using normal donor peripheral blood T cells as starting material. These were stimulated with transact, transduced with lentiviral vector encoding D8Fab-41BBζ and cultured in IL7/IL15 before being used for functional testing.

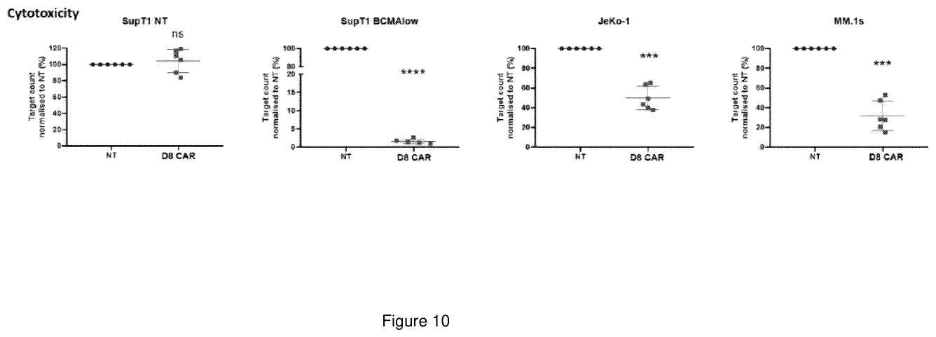

The ability of D8 CAR T cells to lyse target cells was first investigated. As shown in FIG. 10, D8 CAR T cells did not display non-specific killing of antigen-negative SupT1 NT cells but showed effective killing of SupT1 BCMALow cells (1.63±0.64% targets remaining; n=6), JeKo-1 (49.9±11.9% targets remaining; n=6) and MM.1s cells (32.0±15.0% targets remaining; n=6).

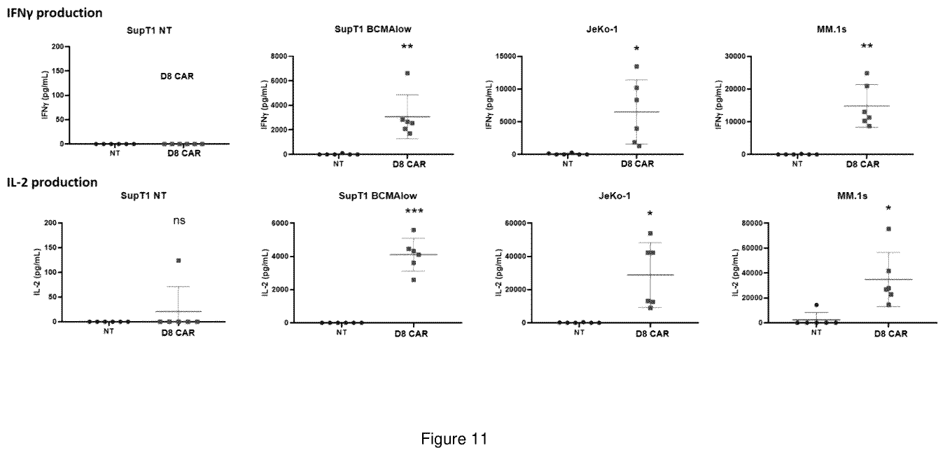

Cytokine production by D8 CAR T cells in response to antigen-expressing target cells was also assessed using culture supernatants from 24-hour co-cultures. As shown in FIG. 11, there was no detectable background production of IFN-γ against antigen-negative SupT1 NT targets by D8 CAR T cells. In co-cultures with SupT1 BCMAlow, D8 CAR T cells produced a mean of 3079±1787 μg/mL of IFN-γ (n=6). With JeKo-1 target cells, D8 CAR T cells produced a significant amount of 6508±4905 μg/mL of IFN-γ whereas with MM.1s cells, D8 CAR T cells produced around 14843±6519 μg/mL of IFN-γ (n=6).

IL-2 production of D8 CAR T cells mirrors the cytotoxicity and IFN-γ production capacity seen in the same co-cultures. Negligible amounts of IL-2 was produced by D8 CAR T cells against SupT1 NT in one donor (FIG. 11), whereas with SupT1 BCMAlow, an average of 4111±992.1 pg/mL of IL-2 (n=6) were produced in the 24 hour co-culture. Higher amounts of IL-2 were produced in co-cultures with JeKo-1 and MM.1s targets cells, at 28853±19459 μg/mL and 34820±21763 μg/mL (n=6) respectively.

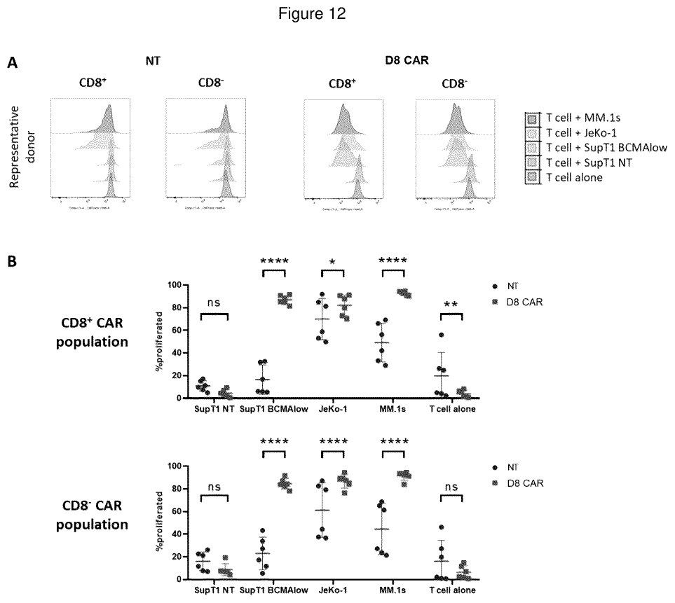

Proliferation of D8 CAR T cells were also investigated. D8 CAR T cells and non-transduced T cells were co-cultured with antigen-negative SupT1 NT, or SupT1 BCMAlow, JeKo-1 and MM.1s (FIG. 12). Cultures of CAR T cells alone were also included to ex-amine background/tonic activity. T cells were stained with a proliferation tracker dye CTV before assay setup, and dilution of the CTV occurs as cells divide where the dye is diluted down from the parent to the daughter cells resulting in a reduction of the measured fluorescence intensity of the dye.

From co-cultures of BCMA-CAR T cells with SupT1 NTs, and in CAR T cell alone cultures, there was minimal proliferation of BCMA-CAR, indicating minimal non-specific activity of BCMA-CAR T cells. In co-cultures with SupT1 BCMAlow targets, BCMA-CAR T cells showed significant proliferation at 96 hours for both CD8+ and CD8− CAR T cell populations (p<0.0001, n=6). As JeKo-1 is a B-cell lymphoblastic cell line which ex-presses costimulatory molecules including CD80 and CD86, NT T cells proliferated in co-cultures with JeKo-1, however proliferation induced by the BCMA-CAR was still significantly higher compared to NT T cells. Similar results were seen with MM.1s cells, where proliferation of BCMA-CAR T cells was significantly higher than that of NT T cells (p<0.0001, n=6).

### Example 2

Functional comparison of D8 BCMA-CAR against bb2121 CAR and LCAR-B38M

D8 CAR T cells were compared against two previously described anti-BCMA CARs namely bb2121 and LCAR-B38M. Notably, these CARs have been developed as Ide-cel and Cilta-cel respectively.

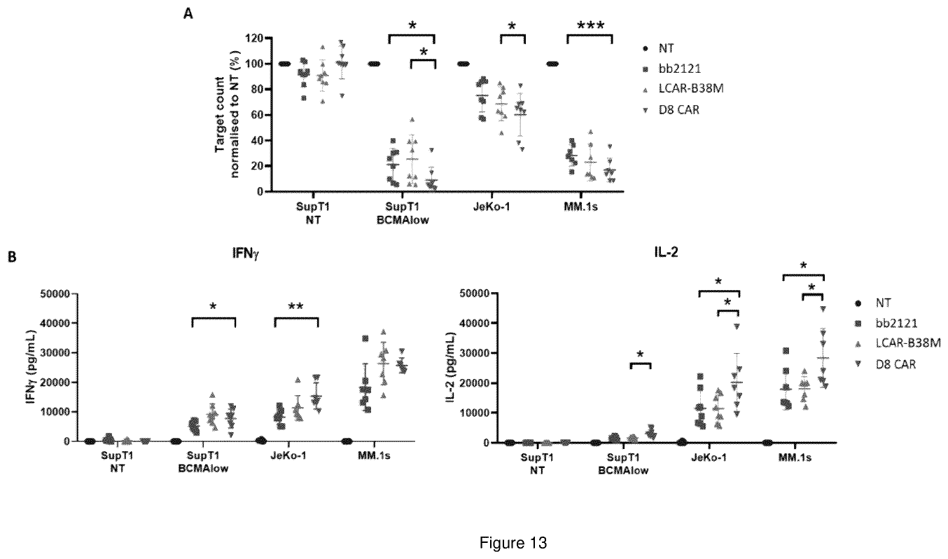

CAR functionality of D8 CAR T cells, bb2121 and LCAR-B38M was first compared by assessing cytotoxicity and cytokine production of these CARs in co-cultures with BCMA-expressing targets. As shown in FIG. 13, there was minimal non-specific killing of anti-gen negative SupT1 NT targets by any CAR T cells. With low BCMA density targets, SupT1 BCMAlow and JeKo-1 cells, D8 CAR T cells showed significantly higher killing of these targets; for example, only 9.1±10.2% of SupT1 BCMAlow targets remained in co-cultures with D8 CAR T cells, whereas 21.1±12.8 and 25.5±19.1% of targets remained in co-cultures with bb2121 or LCAR-B38M CAR T cells respectively. D8 CAR T cells also displayed better cytotoxicity against MM.1s targets, where targets remaining after 24 hours in co-culture with D8 BCMA-CAR was significantly lower than that with bb2121 CAR (p<0.001; n=8).

As regards cytokine release, D8 CAR T cells produced significantly higher levels of IFNγ compared to bb2121 against SupT1.BCMAlow and JeKo-1 cells, whereas production of IFNγ was comparable between D8 and LCAR-B38M. D8 CAR T cells produced significantly more IL-2 compared to the other two CARs in co-cultures with BCMA-expressing targets. For instance, D8 CAR T cells produced IL-2 levels of 28380±9802 μg/mL in co-cultures with MM.1s cells, which was higher compared to levels produced by bb2121 or LCAR-B38M (17996±7019 and 18144±4053 μg/mL respectively).

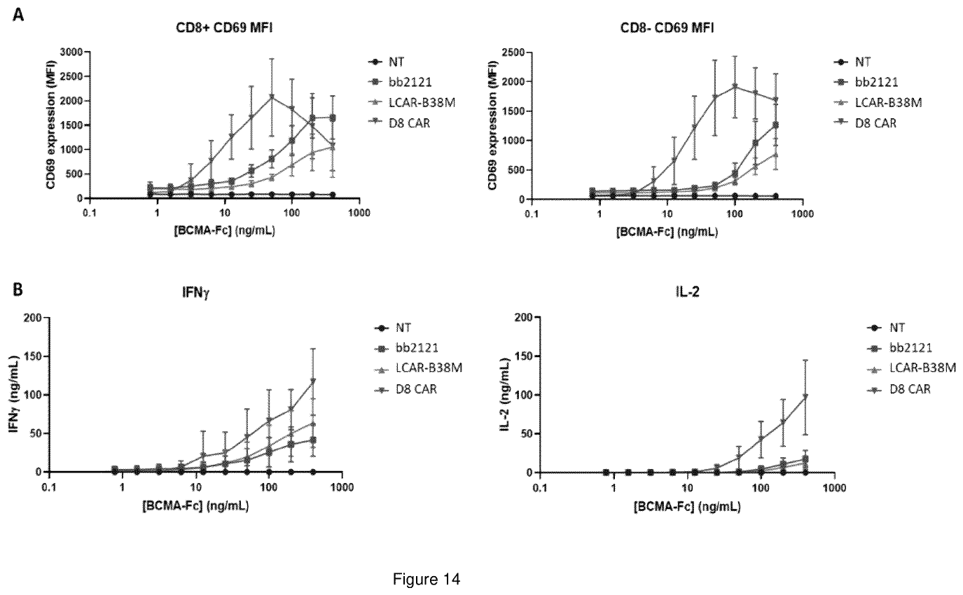

To compare sensitivity to antigen density more finely, CAR T cells were challenged with dilutions of plate bound antigen. CAR T cells were plated on tissue culture plates which had been previously incubated with different concentrations of BCMA-Fc and washed. After 24 hours, the expression of T cell activation marker CD69 and release of cytokines IFNγ and IL-2 were measured and compared. As shown in FIG. 14, D8 CAR T cells showed higher upregulation of CD69 expression to lower amounts of plate-bound BCMA compared to bb2121 and LCAR-B38M CAR T cells. D8 CAR T cells also produced more IFNγ and IL-2 in response to lower amounts of plate-bound antigen stimulation compared to bb2121 and LCAR-B38M CAR T cells.

### Example 3

### Expression and Characterization of D81CAT CAR-T Cells

D8/CAT CAR T cells are generated by transducing patient derived T cells with two lentiviral vectors which encode D8Fab-41BB-ζ and CAT-41BB-ζ. The product is complex comprising of T cells which express either or both CARs at different stoichiometries.

### Expression of D8/CAT in Double-Transduced T Cells

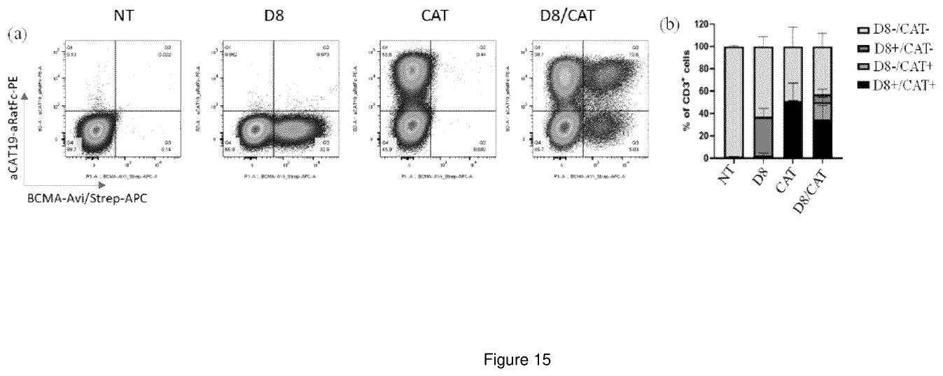

CAR T cells are generated from normal donor PBMCs as described above, but transduced with a mixture of two vectors at a multiplicity of infection (MOI) of 2.5 for each vector. FIG. 15 shows an example flow cytometry plots from a representative double transduction and a stacked bar graph showing mean percentage of D8−CAT−, D8+CAT−, D8+CAT+ and D8-CAT+ cells within the CD3+ population of the transduced PBMCs (n=9).

### In Vitro Function of D8/CAT CAR T Cells

To demonstrate the cytotoxicity of D8/CAT CAR T cells against BCMA-expressing and CD19-expressing targets, co-cultures with different targets and effectors were performed. Effectors were D8 CAR T cells, CAT CAR T cells and D8/CAT CAR T cells. Targets were SupT1 cells (negative for both BCMA and CD19), SupT.BCMAlow, SupT1.CD19 (SupT1 cells engineered to express CD19) and SupT1 BCMAlow.CD19 (SupT1.BCMAlow additionally engineered to express CD19). After a 96-hour incubation period, killing was determined by flow-cytometry.

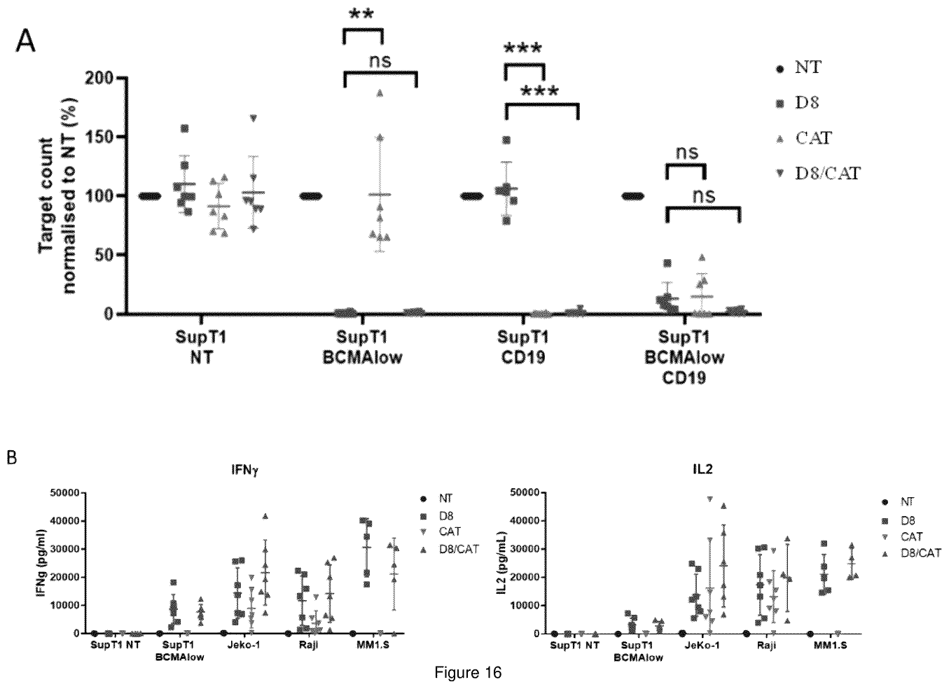

As shown in FIG. 16, SupT1 BCMAlow targets were killed by D8 and D8/CAT CAR T cells but not by CAT CAR T cells. Conversely, SupT1 CD19 targets were killed by CAT and D8/CAT CAR T cells but not by D8 CAR T cells. SupT1.BCMAlow.CD19 targets were killed by D8, CAT and D8/CAT CAR T cells. For assessment of cytokine production CAR T cells were cul-tured with antigen-negative SupT1 NT cells, BCMA-expressing SupT1-BCMAlow and MM1.S cells and BCMA/CD19 dual positive Raji and Jeko-1 cells. As expected, CAT CAR T cells only showed cytokine production against CD19-expressing Raji and Jeko-1 cells, D8 CAR T cells demonstrated cytokine production against BCMA-expressing cells lines, whilst D8/CAT dual CAR-expressing T cells demonstrated cytokine secretion against all BCMA-expressing lines regardless of the presence or absence of CD19.

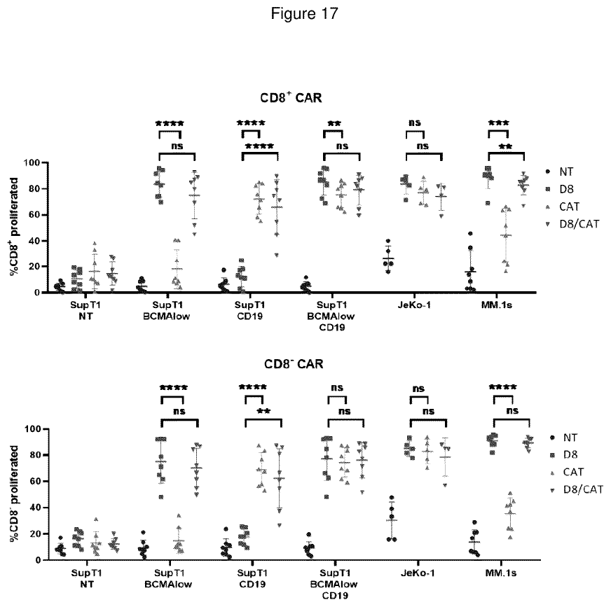

Proliferation of D8/CAT CAR T cells in response to target cells was also tested. CAR-transduced T cells were labelled with a proliferation tracker dye CTV before co-culturing with target cell lines expressing either BCMA or CD19 alone or co-expressing both anti-gens. Please note that JeKo-1 cells are CD19+. D8/CAT CAR T cells showed significant proliferation against target cell lines which express BCMA (SupT1 BCMAlow, SupT1 BCMAlow CD19, JeKo-1 and MM.1s) which were comparable to that seen with D8 CAR T cells (FIG. 17). Furthermore, D8/CAT CAR T cells were capable of proliferating against SupT1 which are expressing CD19 alone whereas D8 single transduced T cells did not, indicative of the proliferative function of CAT19 CAR against CD19 antigen.

### Example 4

### In Vivo Testing of D8 and D8/CAT CAR T Cells

To investigate the in vivo efficacy of D8 and D8/CAT CAR T cells, we decided to utilize Jeko1 cells in an NSG mouse model. In clinical trials involving CAR T cells against B cell malignancies tumour cells expressing low levels of target antigen are seen to escape CAR T surveillance and may be responsible for antigen positive relapse. Therefore, the ability of the CAR to recognize and kill tumour cells expressing low levels of the target antigen is likely to be key to deep and durable responses in the patient and for the avoidance of antigen-positive relapses. Jeko1 express the lowest level of BCMA amongst the target cells we had used and therefore recapitulate a scenario in which the myeloma cells in a patient express very low levels of BCMA.

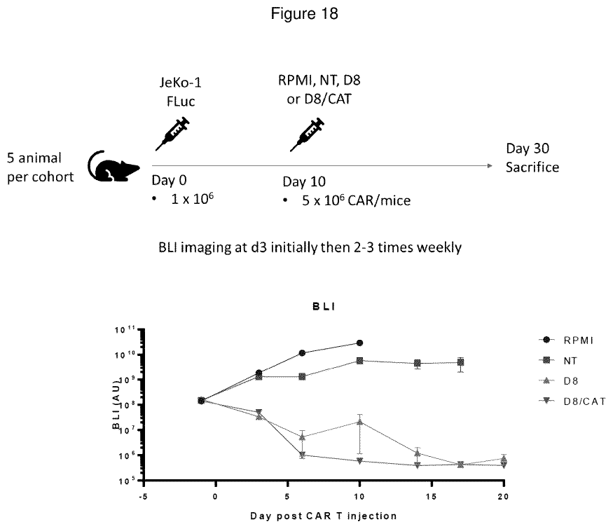

FIG. 18 shows the summary of the experimental design and the results. Briefly, NSG mice were injected with 1×10⁶ Fluc expressing JeKo-1 cells at DO and were allowed to engraft in the mice for the following 10 days. T cells were transduced at a total MOI of 5 and in the presence of 2.5 uM AKTi VIII and 5×10⁶ CAR expressing T cells were subsequently injected into the mice at D10. Mice were then imaged 3 times a week and then sacrificed at D30. There was clear evidence of tumour regression in CAR-treated groups with a trend to a faster anti-tumour response in the mice receiving dual-transduced D8/CAT CAR T cells compared to D8 alone although both cohorts reached a similar level of tumour regression by the endpoint of the model.

Therefore, in the preclinical evaluation of the ATIMPs in MCARTY, D8 was shown to bind selectively without cross reactivity to normal tissues. In a CAR format, the greater sensitivity of the D8 in a Fab vs scFv format to BCMA expressing targets was demon-strated. Further, efficacy of D8 CAR T cells was favourable compared to bb2121 (ide-cel) and LCAR-B38M (cilta-cel) by kill and cytokine release with cells expressing low levels of BCMA and in the presence of soluble BCMA respectively. D8/CAT CARs were shown to maintain killing and proliferation on coculture with CD19 and BCMA expressing targets compared to T cells expressing either CAR alone. We demonstrated the increased frequency of naïve phenotypes with addition of AKTi VIII during CAR manufacture and improved expansion with serial stimulation in vitro. Finally, the in vivo efficacy of D8 and D8/CAT was demonstrated in a xenogeneic murine myeloma model.

### Example 5

### Clinical Study

A study of the safety, efficacy and duration of response of BCMA D8 FabCAR alone and of CAR T cells engineered to co-express the BCMA CAR and a CD19 CAR in patients with relapsed/refractory Multiple Myeloma. This was a Phase 1 rolling 6 trial design evaluating safety of a novel BCMA CAR alone and of CAR T cells engineered to co-express BCMA CAR and a CD19 CAR in patients with triple refractory Multiple Myeloma. Cohort 1 assesses treatment with D8 CAR-T cells (autologous BCMA CAR-T cells) at 50 and 150×10\hat{ }6 cells. Cohort 2 assesses treatment with a combined D8/CAT CAR-T cells (autologous BCMA/CD19 CAR-T cells) and has 2 potential doses of 50 and 150×10\hat{ }6 cells.

The study design is summarized in the following Table 5.

TABLE 5

| Arm | Intervention/treatment |
|---|---|
| Experimental: Cohort 1: BCMA CAR T cells | Biological: BCMA CAR T cells |
| Treatment with Advanced Therapy Investigational | Infusion with ATIMP: BCMA CAR |
| Product (ATIMP): BCMA CAR T-cells | T-cells |
| Experimental: Cohort 2: BCMA/CD19 CAR T cells | Biological: BCMA/CD19 CAR T |
| Treatment with Advanced Therapy Investigational | cells |
| Product (ATIMP): BCMA/CD19 CAR T-cells | Infusion with ATIMP: BCMA/CD19 |
| | CAR T-cells |

The primary outcome measures for the study are as follows:
1. Toxicity of D8 CAR T cells or D8/CAT CAR T cells as evaluated by the incidence of grade 3-5 toxicity causally related to the Advanced Therapy Investigational Product (Advanced Therapy Investigational Me-dicinal Product, ATIMP) [Time Frame: 28 days]

The incidence of grade 3-5 toxicity assessed using the Common Terminology Criteria for Adverse Events (CTCAE) v5.0 and the American Society for Transplantation and Cellular Therapy (ASTCT) Cytokine Release Syndrome (CRS) and Neurotoxicity tool
2. Feasibility of manufacturing CAR T-cells (ATIMP) evaluated by the number of therapeutic products generated [Time Frame: 30 days]

Feasibility of generation of CAR T cells as evaluated by the number of therapeutic products generated.

The secondary outcome measures for the study are as follows.
Best objective response rate (ORR, i.e. ≥partial response, PR, very good partial response, VGPR as defined by IMWG criteria)
Overall rate of CR/sCR
Overall rate of MRD-negative (10-5) response
Rate of CR with MRD-negativity at 12±3 months
Duration of response in patients achieving ≥PR
Median PFS and PFS at 1 year
OS at 1 year
Incidence and severity of adverse events
Occurrence of neurotoxicity, early and late considered related to CAR T cells
Incidence and duration of hypogammaglobulinaemia (all cohorts) and B-cell aplasia by flow cytometry of peripheral blood (cohort 2) Additional exploratory endpoints for the study are as follows.
Sustained MRD negative (10⁻⁵) response (≥12 months)
Expansion and persistence of CAR T cells in bone marrow (BM)
Persistence of circulating CAR T cells by PCR and FACS assessment of peripheral blood
Cytokine levels in peripheral blood
Assessment of tumour BCMA expression and soluble BCMA level in blood
Assessment of CD19 expressing cells in BM, including MM tumour cells
Phenotype of CAR T cells in peripheral blood (PB) and BM.

The inclusion criteria for the study are as follows.
1. Age≥18
2. Relapsed/Refractory Multiple Myeloma
3. Secretory disease: PP≥5 g/L and/or sFLC≥100 mg/L of involved light chain with abnormal K:L ratio.
4. ≥3 prior lines of therapies (including proteasome inhibitor, IMiD, anti CD38 antibody)
5. Refractory to last line of therapy (not achieved at least PR and progressed within 60 days of last dose or achieved at least PR but progressed within 6 months of last dose)
6. Has previously received or is not suitable for ASCT
7. Eastern Cooperative Oncology Group (ECOG) performance status 0/1
8. Creatinine Clearance (CrCl)≥60 ml/min, Absolute Neutrophil Count (ANC)≥1×10\hat{ }9/L, Platelets (plt)≥50×10\hat{ }9/L, Haemoglobin (Hb)≥80/L, lymphocyte count 20.3×10\hat{ }9/L

Exclusion Criteria for registration are as follows.
1. Previous diagnosis of systemic light chain amyloidosis
2. Prior treatment with investigational or approved gene therapy or cell therapy products or allogenic stem cell transplant will be excluded
3. Stem cell transplant patients only:
   allogeneic stem cell transplant within 12 months prior to registration into the study
   moderate/severe chronic GVHD (NIH consensus criteria) requiring immunosuppressive therapy and/or systemic steroids
4. Oxygen saturation ≤90% on air
5. Patients with clinically significant, uncontrolled heart disease or a recent (within 6 months) cardiac event
6. Left ventricular ejection fraction <50% (ECHO or MUGA)
7. Corrected QT interval (QTc)>470 ms on ECG
8. Uncontrolled cardiac arrhythmia (patients with rate-controlled atrial fibrillation are not excluded)
9. History or evidence of deep vein thrombosis or pulmonary embolism requiring ongoing therapeutic anticoagulation at preconditioning
10. Chronic renal impairment requiring dialysis, or creatinine clearance <60 ml/min
11. Patients with significant liver disease: alanine aminotransferase or aspartate aminotransferase 23× upper limit normal (ULN), or total bilirubin≥25 umol/L (1.5 mg/dL), except in patients with Gilbert's syndrome, or evidence of end-stage liver disease (e.g. ascites, hepatic encephalopathy)
12. Patients with any major surgical intervention in the last 3 months, cement augmentation for vertebral collapse is permitted
13. Patients with active gastrointestinal bleeding
14. Patients with active infectious bacterial or viral disease requiring treatment
15. Known active central nervous system involvement of MM. History or presence of clinically relevant central nervous system pathology such as epilepsy, paresis, aphasia, stroke within 3 months prior to enrolment, severe brain injuries, dementia, Parkinson's disease, cerebellar disease, organic brain syndrome, uncontrolled mental illness, or psychosis
16. Patients receiving corticosteroids at a dose of >5 mg prednisolone per day (or equivalent) that cannot be discontinued
17. Use of rituximab (or rituximab biosimilar) within the last 3 months prior to CAR T-cell infusion
18. Active autoimmune disease requiring immunosuppression
19. Past or current history of other neoplasms
20. Received any radiotherapy within the last 7 days prior to lymphodepletion or leukapheresis. Localised radiation to a single site, e.g. for bone pain is permitted at any time
21. Patients with any anti-myeloma therapy within the last 7 days prior to LD or leukapheresis
22. Inability to tolerate leucapheresis
23. Life expectancy <3 months
24. Women who are pregnant or breastfeeding
25. Known allergy to albumin or DMSO

The exclusion criteria for BCMA/CD19CAR T-cell infusion are as follows.
1. Active infection requiring systemic anti-microbial therapy, or with temperature more or equal to 38 C within 48 hours before scheduled CAR-T cell infusion
2. Requirement for supplementary oxygen at the time of scheduled CAR-T cell infusion
3. Clinical deterioration of organ functions (hepatic or renal function) exceeding criteria set at study entry

### Results

### Patient Characteristics

Eight patients have been recruited onto the trial and 6 patients have been treated (5 with BCMA CAR T cells and 1 with BCMA/CD19 CAR T cells). Data relevant to the 6^{th} patient (MCA-01-07), who is the first patient to be treated with the BCMA/CD19 CAR, is marked with ‘*’ in Table 6.

TABLE 6
Baseline characteristics for treated patients.

| Characteristic | N = 6 |
|---|---|
| Age (years), median (range) | 60.5 (33-66) |
| *MCA-A01-07 aged 65 | |
| Sex | |
| Female | 2* (33) |
| Male | 4 (67) |
| Type of myeloma | |
| Secretory | 6* (100) |
| Stage of disease at diagnosis | |
| I | 1 (17) |
| II | 2 (33) |
| III | 2* (33) |
| Not known | 1 (17) |
| Previous treatment details | |
| Prior lines, median (range) | 4 (3-8) |
| *MCA-A01-07 5 prior lines | |
| Prior surgery for MM | 0 |
| Radiotherapy | 1 (17) |
| ASCT | 6* (100) |
| Serum paraprotein expression at registration | |
| Single paraprotein | 4* (67) |
| Biclonal | 2 (33) |
| Myeloma evidence on imaging (CT, PET-CT or MRI) | |
| Yes | 5* (83) |
| No | 1 (17) |
| ECOG | |
| 0 | 4 (67) |
| 1 | 2* (33) |

It was possible to manufacture successfully the ATIMP (CAR-T cell product) at the desired CAR-T cell dose for cohort 1 (50 and 150×10\hat{ }6 CAR T cells, BCMA CAR-T cells) and cohort 2 (50×10\hat{ }6 CAR T cells, BCMA/CD19 CAR-T cells). It is possible to manufacture the ATIMP (CAR-T cell product) at the desired CAR-T cell dose for cohort 2 (150×10\hat{ }6 CAR T cells, BCMA/CD19 CAR-T cells).

### Toxicity Assessment

Toxicity evaluation following D8 or D8/CAT CAR T cells administration is a primary endpoint for MARTY. This is assessed by the occurrence of adverse events (AEs) grade 3-5 casually related to the ATIMP.

The summary of the maximum grade of the AEs reported for the 3 patients treated with 5×10\hat{ }6 BCMA CAR T cells is listed in Table 7 [Cohort 1 (lower dose): 50×10\hat{ }6 BCMA CAR T cells]. No grade 5 events were reported.

TABLE 7

| | Infused patients (N = 3) | | | |
|---|---|---|---|---|
| Adverse events | Grade 1 | 2 | 3 | 4 |
| Blood and lymphatic system disorders | 0 | 0 | 2 (67) | 0 |
| Anaemia | 0 | 0 | 1 (33) | 0 |
| Febrile neutropenia | 0 | 0 | 2 (67) | 0 |
| Gastrointestinal disorders | 3 (100) | 0 | 0 | 0 |
| Abdominal pain | 1 (33) | 0 | 0 | 0 |
| Diarrhoea | 2 (67) | 0 | 0 | 0 |
| Hemorrhoids | 1 (33) | 0 | 0 | 0 |
| Nausea | 1 (33) | 0 | 0 | 0 |
| General disorders and administration site conditions | 0 | 2 (67) | 1 (33) | 0 |
| Edema limbs | 1 (33) | 0 | 0 | 0 |
| Fatigue | 2 (67) | 0 | 0 | 0 |
| Fever | 1 (33) | 1 (33) | 1 (33) | 0 |
| Pain | 0 | 1 (33) | 0 | 0 |
| Infections and infestations | 1 (33) | 0 | 0 | 0 |
| Other: COVID Positive | 1 (33) | 0 | 0 | 0 |
| Investigations | 0 | 0 | 0 | 3 (100) |
| Alanine aminotransferase increased | 0 | 0 | 1 (33) | 0 |
| Alkaline phosphatase increased | 0 | 1 (33) | 0 | 0 |
| Neutrophil count decreased | 0 | 0 | 0 | 3 (100) |
| Platelet count decreased | 0 | 1 (33) | 0 | 1 (33) |
| Metabolism and nutrition disorders | 0 | 0 | 1 (33) | 0 |
| Hypoalbuminemia | 0 | 1 (33) | 0 | 0 |
| Hypophosphatemia | 0 | 0 | 1 (33) | 0 |
| Musculoskeletal and connective tissue disorders | 1 (33) | 0 | 0 | 0 |
| Bone pain | 1 (33) | 0 | 0 | 0 |
| Nervous system disorders | 3 (100) | 0 | 0 | 0 |
| Headache | 3 (100) | 0 | 0 | 0 |
| Non-CTCAE | 1 (33) | 2 (67) | 0 | 0 |
| Cytokine Release Syndrome (CRS) | 1 (33) | 2 (67) | 0 | 0 |
| Renal and urinary disorders | 1 (33) | 0 | 0 | 0 |
| Urinary frequency | 1 (33) | 0 | 0 | 0 |
| Respiratory, thoracic and mediastinal disorders | 0 | 1 (33) | 0 | 0 |
| Cough | 1 (33) | 0 | 0 | 0 |
| Hypoxia | 0 | 1 (33) | 0 | 0 |
| Rhinorrhea | 1 (33) | 0 | 0 | 0 |

Table 8 summarizes the maximum grade of AEs for all patients treated with 150×10A6 BCMA CAR T cells [Cohort 1 (higher dose): 150×10\hat{ }6 BCMA CAR T cells]. No grade 5 events reported.

TABLE 8

| | Infused patients (N = 2) | | | |
|---|---|---|---|---|
| Adverse event | Grade 1 | 2 | 3 | 4 |
| Blood and lymphatic system disorders | 0 | 0 | 2 (100) | 0 |
| Anaemia | 0 | 0 | 2 (100) | 0 |
| Febrile neutropenia | 0 | 0 | 1 (50) | 0 |
| Gastrointestinal disorders | 1 (50) | 1 (50) | 0 | 0 |
| Diarrhoea | 1 (50) | 1 (50) | 0 | 0 |
| General disorders and administration site conditions | 2 (100) | 0 | 0 | 0 |
| Chills | 1 (50) | 0 | 0 | 0 |
| Fatigue | 1 (50) | 0 | 0 | 0 |
| Fever | 2 (100) | 0 | 0 | 0 |
| Infections and infestations | 1 (50) | 0 | 0 | 0 |
| Thrush | 1 (50) | 0 | 0 | 0 |
| Investigations | 0 | 0 | 0 | 2 (100) |
| Neutrophil count decreased | 0 | 0 | 0 | 2 (100) |
| Platelet count decreased | 0 | 0 | 2 (100) | 0 |
| Metabolism and nutrition disorders | 0 | 0 | 2 (100) | 0 |
| Anorexia | 0 | 1 (50) | 0 | 0 |
| Hypoalbuminemia | 0 | 1 (50) | 0 | 0 |
| Hypokalemia | 0 | 2 (100) | 0 | 0 |
| Hypophosphatemia | 0 | 0 | 2 (100) | 0 |
| Nervous system disorders | 1 (50) | 0 | 0 | 0 |
| Headache | 1 (50) | 0 | 0 | 0 |
| Non-CTCAE | 1 (50) | 1 (50) | 0 | 0 |
| Cytokine Release Syndrome (CRS) | 1 (50) | 1 (50) | 0 | 0 |
| Psychiatric disorders | 1 (50) | 0 | 0 | 0 |
| Anxiety | 1 (50) | 0 | 0 | 0 |
| Depression | 1 (50) | 0 | 0 | 0 |
| Vascular disorders | 0 | 1 (50) | 0 | 0 |
| Hypotension | 0 | 1 (50) | 0 | 0 |

Table 9 summarizes the maximum grade of AEs for all patients treated with 50×10\hat{ }6 BCMA/CD19 CAR T cells [Cohort 2 (lower dose): 50×10\hat{ }6 D8/CAT CAR T cells]. No grade 5 events reported.

TABLE 9

| | Infused |
|---|---|
| | patients (N = 1) |
| Adverse Event | Max grade |
| Anaemia | 3 |
| Atrial fibrillation | 2 |
| Cytokine Release Syndrome (CRS) | 1 |
| Diarrhoea | 2 |
| Febrile neutropenia | 3 |
| Fever | 2 |
| Hypocalcemia | 2 |
| Hypophosphatemia | 2 |
| Lymphocyte count decreased | 4 |
| Neutrophil count decreased | 4 |
| Other: Cardiomegaly | 2 |
| Platelet count decreased | 4 |
| White blood cell decreased | 4 |

This patient developed Grade 1 CRS on the day of infusions with febrile episodes lasting until D+18. She did not receive tocilizumab. At D+12 there was also evidence of pneumonia on imaging which was thought the primary cause of a short and transient requirement for supplemental oxygen. Also during this time, the patient had periods of fast atrial fibrillation and remained cardiovascular stable during these episodes. A subsequent echo at D+23 was suggestive of moderate LVEF (estimated 40-45%) which was thought by Cardiology to represent some stress cardiomyopathy and this ECHO will be repeated in due course. As of D+28 this patient requires transfusion support with blood and platelets and has been started in GCSF. Nevertheless, this patient tolerated CAR T cells well.

Furthermore, there were no dose limiting toxicities (DLTs) related to the CAR-T cell product. DLT will be defined as any of the following CAR T cell related adverse events which occur within the DLT period (between DO and D28 of CAR T cell infusion):
Any new non-haematological AE of Grade 3 or higher (using CTCAE v 5.0) which fails to resolve to Grade 2 or better within 14 days, despite appropriate supportive measures;
A Grade 4 CRS, neurotoxicity (ICANS), or cerebral oedema (using ASTCT criteria for CRS & ICANS);
Grade 3 ICANS that lasts >72 hours (using ASTCT criteria for neurotoxicity);
Grade >2 Infusion Reaction with CAR T infusion;
Any other fatal event (Grade 5), or life-threatening event (Grade 4) that cannot be managed with conventional supportive measures, or which necessitates modification to trial treatment to avoid a similar occurrence in future patients; and
Any event that in the opinion of the TMG put patient at undue risk may also be considered a DLT.

Moreover, there were no AEs of special interest (Grade 3-5 CRS, Grade 3-5 Neurotoxicity/ICANS) reported.

Therefore, BCMA CAR T cell product (D8 CAR-T cells) and the BCMA/CD19 CAR T cell product (D8/CAT CAR-T cells) showed good safety profiles.

### Efficacy

Patients had good overall response rate (as defined in the secondary outcome measures). Table 10 summarizes the responses of all treated patients.

TABLE 10

| | | | Last known | Time of last |
|---|---|---|---|---|
| Patient | Dose | Cohort | response | known response |
| 01 | 50 | D8 CAR-T | sCR | Month 6 |
| 02 | 50 | D8 CAR-T | CR | Month 6 |
| 03 | 50 | D8 CAR-T | PR | Month 4 |
| 04 | 150 | D8 CAR-T | CR | Month 2 |

| | | | |
|---|---|---|---|
| 05 | 150 | D8 CAR-T | No response data yet |
| 07 | 50 | D8/CAT CAR-T | No response data yet |

Notably, no progressions or deaths have been reported to date.

### Additional Exploratory Endpoints

The BCMA/CD19 CAR-T cell product shows high expansion and persistence.

Sustained MRD negative (10⁻⁵) response (≥12 months) is high.

Expansion and persistence of CAR T cells in bone marrow (BM) is high.

Persistence of circulating CAR T cells by PCR and FACS assessment of peripheral blood is high.

1. A method of treating relapsed or resistant BCMA+ or CD19+ malignancy in a patient comprising administering autologous BCMA/CD19 CAR T-cells to the patient.

2. (canceled)

3. The method of claim 1 wherein the BCMA+ or CD19+ malignancy is multiple myeloma (MM) or a BCMA+ or CD19+ plasma cell disorder.

4. The method of claim 3 wherein the BCMA+ or CD19+ malignancy is multiple myeloma (MM).

5. The method of claim 1 wherein the patient has:
a) Relapsed or refractory Multiple Myeloma;
b) Secretory disease;
c) ≥3 prior lines of therapy;
d) Refractory to last line of therapy; or
e) Has previously received or is not suitable for autologous stem cell transplant (ASCT).

6. The method of claim 1 wherein the patient is administered a single dose of 5×10⁶ CAR T-cells, 25×10⁶ CAR T-cells, 50×10⁶ CAR T-cells, or 150×10⁶ CAR T-cells.

7. The method of claim 6 wherein the administration is an intravenous injection through a Hickman line or peripherally inserted central catheter.

8. The method of claim 1 wherein the BCMA/CD19 CAR T-cell product expresses a chimeric antigen receptor (CAR) comprising a BCMA-binding domain which comprises
a) a heavy chain variable region (VH) having CDRs with the following sequences:
(SEQ ID NO: 1)
CDR1-GFIFSDYN
(SEQ ID NO: 2)
CDR2-IIYDGSST
(SEQ ID NO: 3)
CDR3-ATRPGPFAY;
and
b) a light chain variable region (VL) having CDRs with the following sequences:
```
  (SEQ ID NO: 4)
CDR1-QSLLHSNGNTY
  (SEQ ID NO: 5)
CDR2-LVS
(SEQ ID NO: 6)
CDR3-VHGTHAWT.
```

9. The method of claim 8, wherein the BCMA-binding domain comprises a VH domain having the sequence shown as SEQ ID NO: 7 and/or or a VL domain having the sequence shown as SEQ ID NO: 8 or a variant thereof having at least 95% sequence identity.

10. The method of claim 8, wherein the BCMA-binding domain comprises an scFv in the orientation VH-VL.

11. The method of claim 8, wherein the BCMA-binding domain comprises a Fab.

12. The method of claim 11, wherein the BCMA-binding Fab comprises a heavy chain (VH-CH1) sequence shown as SEQ ID NO: 9.

13. The method of claim 11, wherein the BCMA-binding Fab comprises a light chain (VL-CL kappa) sequence shown as SEQ ID NO: 10.

14-18. (canceled)

19. The method of claim 1 wherein the BCMA/CD19 CAR T-cell product expresses a chimeric antigen receptor (CAR) comprising a CD19-binding domain which comprises
a) a heavy chain variable region (VH) having complementarity determining regions (CDRs) with the following sequences:
```
  (SEQ ID NO: 37)
CDR1-GYAFSSS;
  (SEQ ID NO: 38)
CDR2-YPGDED
  (SEQ ID NO: 39)
CDR3-SLLYGDYLDY;
```
and
b) a light chain variable region (VL) having CDRs with the following sequences:
```
  (SEQ ID NO: 40)
CDR1-SASSSVSYMH;
  (SEQ ID NO: 41)
CDR2-DTSKLAS
(SEQ ID NO: 42)
CDR3-QQWNINPLT.
```

20. The method of claim 19, wherein the CD19-binding domain comprises a VH domain having the sequence shown as SEQ ID NO: 43 and/or or a VL domain having the sequence shown as SEQ ID NO: 44 or a variant thereof having at least 95% sequence identity.

21. The method of claim 19, wherein the CD19-binding domain comprises an scFv in the orientation VH-VL.

22. The method of claim 21, wherein the CD19-binding domain comprises the sequence shown as SEQ ID NO: 45 or a variant thereof having at least 90% sequence identity.

23-27. (canceled)

28. The method of claim 1 wherein the autologous CD19/22 CAR T-cells comprise the BCMA/CD19 CAR T-cell product comprising D8 FabCAR and CAT19CAR CARs.


SEQUENCE LISTING (94 sequences)

SEQ ID NO: 1 - protein, 8 aa, Homo sapiens
  source 1..8: note = Heavy chain
GFIFSDYN

SEQ ID NO: 2 - protein, 8 aa, Homo sapiens
  source 1..8: note = Heavy chain
IIYDGSST

SEQ ID NO: 3 - protein, 9 aa, Homo sapiens
  source 1..9: note = Heavy chain
ATRPGPFAY

SEQ ID NO: 4 - protein, 11 aa, Homo sapiens
  source 1..11: note = Light chain
QSLLHSNGNTY

SEQ ID NO: 5 - skipped

SEQ ID NO: 6 - protein, 8 aa, Homo sapiens
  source 1..8: note = Heavy chain
VHGTHAWT

SEQ ID NO: 7 - protein, 115 aa, Homo sapiens
  source 1..115: note = Heavy chain
EVQLVESGGGLVQPGRSLKLSCAASGFIFSDYNMAWVRQAPKKGLEWVATIIYDGSSTNHGDSVKGRFTISRDNAKSTLYLQMDSLRSEDTATYYCATRPGPFAYWGQGTLVTVS

SEQ ID NO: 8 - protein, 111 aa, Homo sapiens
  source 1..111: note = Light Chain
DVVLTQTPPTLSATIGQSVSISCRSSQSLLHSNGNTYLHWLLQRPGQSPQFLIYLVSGLGSGVPNRFSGSGSGTDFTLKISGVEAEDLGIYYCVHGTHAWTVGGGTKLELK

SEQ ID NO: 9 - protein, 214 aa, Homo sapiens
  source 1..214: note = Heavy chain
EVQLVESGGGLVQPGRSLKLSCAASGFIFSDYNMAWVRQAPKKGLEWVATIIYDGSSTNHGDSVKGRFTISRDNAKSTLYLQMDSLRSEDTATYYCATRPGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 10 - protein, 218 aa, Homo sapiens
  source 1..218: note = Light Chain
DVVLTQTPPTLSATIGQSVSISCRSSQSLLHSNGNTYLHWLLQRPGQSPQFLIYLVSGLGSGVPNRFSGSGSGTDFTLKISGVEAEDLGIYYCVHGTHAWTVGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 218 aa, Homo sapiens; identical to SEQ ID NO: 10
  source 1..218: note = Light chain

SEQ ID NO: 12 - protein, 414 aa, Homo sapiens
  source 1..414: note = Heavy chain
EVQLVESGGGLVQPGRSLKLSCAASGFIFSDYNMAWVRQAPKKGLEWVATIIYDGSSTNHGDSVKGRFTISRDNAKSTLYLQMDSLRSEDTATYYCATRPGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 124 aa, Homo sapiens
  source 1..124: note = Heavy chain
QIQLVQSGPELVKPGSSVKLSCKTSGFTFSDSYMSWLKQVPGQSIEWIGNIYAGDGATHYHQKFKGKATLTVDTSSSTAYMDLSSLTSEDSALYFCARPLYTTAYYYVGGFAYWGQGTLVTVSS

SEQ ID NO: 14 - protein, 113 aa, Homo sapiens
  source 1..113: note = Light chain
DIVMTQSPSSLAVSAGETVTINCKSSQSLLSSGNQKNYLAWYQQKPGQSPKLLIYWASTRQSGVPDRFIGSGSGTDFTLTISSVQAEDLAIYYCQQYYDTPLTFGSGTKLEIK

SEQ ID NO: 15 - protein, 121 aa, Homo sapiens
  source 1..121: note = Heavy chain
EVKLVESGGGLVQPGRSLKLSCTASGFTFSNYDMAWVRQAPTKGLEWVASISTSGDTIYYRDSVKGRFTVSRDKAKSTLYLQMDSLRSEDTATYYCARHDYYDGYQSFAYWGQGTLVTVSS

SEQ ID NO: 16 - protein, 107 aa, Homo sapiens
  source 1..107: note = Light chain
NTVMTQSPTSMSISVGDRVTMNCKASQNVGNNIAWYQQKPGQSPKLLIYYASNRYTGVPDRFTGSGSGTDFTLTINSVQAEDAAFYYCQRIYNSALTFGSGTKLEIK

SEQ ID NO: 17 - protein, 120 aa, Homo sapiens
  source 1..120: note = Heavy chain
QVQLQQSGAALVKPGASVKMSCKASGYTFTDYWVSWVKQSHGKSLEWIGEIYPNSGPTNFNKKFKGKATLTVDKSTSTAYMELSRLTSEDSAIYYCTPRTVAPYNWFAYWGQGTLVTVSS

SEQ ID NO: 18 - protein, 106 aa, Homo sapiens
  source 1..106: note = Light chain
DIVLTQSPALAVSPGERVSISCRASESVSTRMHWYQQKPGQQPKLLIYGASNLESGVPARFSGSGSGTDFTLTIDPVEADDTATYFCQQSWNDPYTFGAGTKLELK

SEQ ID NO: 19 - protein, 121 aa, Homo sapiens
  source 1..121: note = Heavy chain
EVQLVESGGGLVQPGRSLKLSCSASGFIFSNFDMAWVRQAPRKGLEWVASITTSGGDTHYRDSVKGRFTVSRHNAKSTLYLQMDSLRSEDTATYYCARHVYYGLFWFFDFWGPGTMVTVSS

SEQ ID NO: 20 - protein, 108 aa, Homo sapiens
  source 1..108: note = Light chain
NTVMTQSPKSIFISVGDRVTVNCKASQNVGTNVDWYQQKTGQSPKLLIYGASNRYTGVPDRFTGSGSGTDFTFTISNMQAEDLAVYYCMQSNTNPFTFGAGTKLELKR

SEQ ID NO: 21 - protein, 121 aa, Homo sapiens
  source 1..121: note = Heavy chain
EVQLVESGGGLVQPGRSLKLSCTASGFTFSNYDMAWVRQAPTKGLEWVASISTSGDTIYYRDSVKGRFTVSRDKAKSTLYLQMDSLRSEDTATYYCARHDYYDGYQSFAYWGQGTLVTVSS

SEQ ID NO: 22 - protein, 108 aa, Homo sapiens
  source 1..108: note = Light chain
DIVMTQSPSTLPASLGERVTISCRASQSISNYLNWYQQKPDGTIKPLIYYTSNLQSGVPSRFSGSGSGTDYSLTISSLEPEDFAMYYCQQDASFPWTFGGGTKLELKR

SEQ ID NO: 23 - protein, 119 aa, Homo sapiens
  source 1..119: note = Heavy chain
EVQLQESGPGLVKPSQSLSLTCSVTGYPITNNYDWSWIRQFPGNKMEWMGYISDSGNTNYNPSLKSRISITRDTSKNQFFLQLNSVTTEDTATYYCASGYISYIPFAFWGQGTLVTVSS

SEQ ID NO: 24 - protein, 111 aa, Homo sapiens
  source 1..111: note = Light chain
DIVLTQSPALAVSLGQRATISCRASQSVSISSYNLMQWYQQKPGQQPKLLIYDASNLASGIPARFSGSGSGTDFTLTIDPVQADDIATYYCQQSKDDPNTFGAGTKLEIKR

SEQ ID NO: 25 - protein, 119 aa, Homo sapiens
  source 1..119: note = Heavy chain
EVQLQESGPGLVQPSQTLSLTCSVTGYPITNNYDWSWIRKFPGNKMEWMGYISDSGSTNYNPSLKSRISITRDTSKNQFFLQLNSVTTEDTATYYCASGYISYIPFGFWGQGTLVTVSS

SEQ ID NO: 26 - protein, 106 aa, Homo sapiens
  source 1..106: note = Light chain
DIVLTQSPALAVSPGERVTISCRASESVSTRMHWYQQKPGQQPKLLIYGASNLESGVPARFSGSGSGTDFTLTIDPVEADDTATYFCQQSWNDPPTFGSGTKLEIK

SEQ ID NO: 27 - protein, 121 aa, Homo sapiens; identical to SEQ ID NO: 21
  source 1..121: note = Heavy chain

SEQ ID NO: 28 - protein, 113 aa, Homo sapiens
  source 1..113: note = Light chain
DIVMTQSPASQAVSAGEKVTMSCKSSQSLLYSGDQKNYLAWYQQKPGQSPKLLIYLASTRESGVPDRFIGSGSGTDFTLTISSVQAEDLADYYCQQHYSYPLTFGSGTKLEIK

SEQ ID NO: 29 - protein, 121 aa, Homo sapiens
  source 1..121: note = Heavy chain
EVQLVESGGGLVQPGRSLKLSCAASGFTFSNYDMAWVRQAPTKGLEWVASISTSGDTIYYRDSVKGRFTVSRDNAKSTLYLQMDSLRSEDTATYYCTRHGYYDGYQSFDYWGQGTLVTVSS

SEQ ID NO: 30 - protein, 107 aa, Homo sapiens
  source 1..107: note = Light chain
NTVMTQSPKSMSISVGDRVTMNCKASQNVGNNIAWYQQKPGQSPKLLIYYASNRYTGVPDRFTGGGYGTDFTLTINSVQAEDAATYYCQQWNYPSITFGSGTKLEIK

SEQ ID NO: 31 - protein, 122 aa, Homo sapiens
  source 1..122: note = Heavy chain
EVQLVESGGGLVQPGRSMKLSCAASGFTFSNYDMAWVRQAPTKGLEWVASISPSGGSTYYRDSVKGRFTVSRDNAKSSLYLQMDSLRSEDTATYYCTRGDYGYNYAYWFAYWGQGTLVTVSS

SEQ ID NO: 32 - protein, 107 aa, Homo sapiens
  source 1..107: note = Light chain
DIVMTQAPSSMPASLGERVTISCRASQGISNYLNWYQQKPDGTIKPLIYYTSNLQSGVPSRFSGSGSGTDYSLTISSLEPEDFAMYYCQQYDSSPLTFGAGTKLELK

SEQ ID NO: 33 - protein, 121 aa, Homo sapiens
  source 1..121: note = Heavy chain
EVQLVESGGGLVQPGRSLKLSCEASGFTFSNYDMAWVRQAPTKGLEWVASISTSGDSIYYRDSVKGRFTVSRDNVKSTLYLQMDSLRSEDTATYYCARHGYYDGYQSFDYWGQGTLVTVSS

SEQ ID NO: 34 - protein, 107 aa, Homo sapiens
  source 1..107: note = Light chain
DIVMTQSPSSLPASLGERVTISCRASQGISNNLNWYQQKPDGTIKPLIYYTSNLQSGVPSRFSGSGSGTDYSLTISSLEPEDFATYYCQQDETFPYTFGAGTKLELK

SEQ ID NO: 35 - protein, 121 aa, Homo sapiens
  source 1..121: note = Heavy chain
EVQLVESGGGLVQPGRSLKLSCAASGFTFSNYDMAWVRQAPTKGLEWVASISPSGGSTYYRDSVKGRFTISRDNAKSTLYLQMDSLRSEDTATYYCATHNYYDGSSLFAYWGQGTLVTVSS

SEQ ID NO: 36 - protein, 106 aa, Homo sapiens
  source 1..106: note = Light chain
DIVLTQSPALAVSPGERVTISCGANETVSTLVHWYQQKPGQQPKLLIYLASHLESGVPARFSGSGSGTDFTLTIDPVEADDTATYYCQQSWNDPPTFGGGTKLELK

SEQ ID NO: 37 - protein, 7 aa, Homo sapiens
  source 1..7: note = Heavy chain
GYAFSSS

SEQ ID NO: 38 - protein, 6 aa, Homo sapiens
  source 1..6: note = Heavy chain
YPGDED

SEQ ID NO: 39 - protein, 10 aa, Homo sapiens
  source 1..10: note = Heavy chain
SLLYGDYLDY

SEQ ID NO: 40 - protein, 10 aa, Homo sapiens
  source 1..10: note = light chain
SASSSVSYMH

SEQ ID NO: 41 - protein, 7 aa, Homo sapiens
  source 1..7: note = Light chain
DTSKLAS

SEQ ID NO: 42 - protein, 9 aa, Homo sapiens
  source 1..9: note = Light chain
QQWNINPLT

SEQ ID NO: 43 - protein, 119 aa, Mus sp.
  source 1..119: note = Heavy chain
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 44 - protein, 107 aa, Mus sp.
  source 1..107: note = Light chain
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 45 - protein, 241 aa, Mus sp.
  source 1..241: note = Heavy/Light chain
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 46 - protein, 488 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 483 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 487 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 527 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 465 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 681 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 5 aa, Homo sapiens
  source 1..5: note = Heavy chain
SYWMN

SEQ ID NO: 53 - protein, 16 aa, Homo sapiens
  source 1..16: note = Heavy chain
QIWPGDGDTNYNGKFK

SEQ ID NO: 54 - protein, 15 aa, Homo sapiens
  source 1..15: note = Heavy chain
RETTTVGRYYYAMDY

SEQ ID NO: 55 - protein, 15 aa, Homo sapiens
  source 1..15: note = Light chain
KASQSVDYDGDSYLN

SEQ ID NO: 56 - protein, 7 aa, Homo sapiens
  source 1..7: note = Light chain
DASNLVS

SEQ ID NO: 57 - protein, 9 aa, Homo sapiens
  source 1..9: note = Light chain
QQSTEDPWT

SEQ ID NO: 58 - protein, 235 aa, Mus sp.
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 59 - protein, 236 aa, Homo sapiens
  source 1..236: note = Heavy chain
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 60 - protein, 236 aa, Homo sapiens
  source 1..236: note = Heavy chain
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 61 - protein, 124 aa, Mus sp.
  source 1..124: note = Heavy chain
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 62 - protein, 124 aa, Homo sapiens
  source 1..124: note = Heavy chain
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 63 - protein, 111 aa, Mus sp.
  source 1..111: note = Light chain
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 64 - protein, 112 aa, Homo sapiens
  source 1..112: note = Light chain
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 65 - protein, 112 aa, Homo sapiens
  source 1..112: note = Light chain
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 66 - protein, 21 aa, Homo sapiens
MGTSLLCWMALCLLGADHADA

SEQ ID NO: 67 - protein, 20 aa, Homo sapiens
METDTLLLWVLLLLVPGSTG

SEQ ID NO: 68 - protein, 20 aa, Homo sapiens
METDTLILWVLLLLVPGSTG

SEQ ID NO: 69 - protein, 19 aa, Homo sapiens
MGWSCIILFLVATATGVHS

SEQ ID NO: 70 - protein, 21 aa, Homo sapiens
MSLPVTALLLPLALLLHAARP

SEQ ID NO: 71 - protein, 20 aa, Homo sapiens
MAVPTQVLGLLLLWLTDARC

SEQ ID NO: 72 - protein, 234 aa, Homo sapiens
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 73 - protein, 45 aa, Homo sapiens
TTTPAPRPPTPAPTIASQPLSLRPEACRPAAGGAVHTRGLDFACD

SEQ ID NO: 74 - protein, 20 aa, Homo sapiens
AEPKSPDKTHTCPPCPKDPK

SEQ ID NO: 75 - protein, 15 aa, Homo sapiens
EPKSCDKTHTCPPCP

SEQ ID NO: 76 - protein, 237 aa, Homo sapiens
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 77 - protein, 236 aa, Homo sapiens
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 78 - protein, 185 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 79 - protein, 259 aa, Homo sapiens
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 80 - protein, 27 aa, Homo sapiens
FWVLVVVGGVLACYSLLVTVAFIIFWV

SEQ ID NO: 81 - protein, 24 aa, Homo sapiens
IIAIAVVGALLLVALIFGTASYLI

SEQ ID NO: 82 - protein, 24 aa, Homo sapiens
IYIWAPLAGTCGVLLLSLVITLYC

SEQ ID NO: 83 - protein, 114 aa, Homo sapiens
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 42 aa, Homo sapiens
KRGRKKLLYIFKQPFMRPVQTTQEEDGCSCRFPEEEEGGCEL

SEQ ID NO: 85 - protein, 37 aa, Homo sapiens
RRDQRLPPDAHKPPGGGSFRTPIQEEQADAHSTLAKI

SEQ ID NO: 86 - protein, 37 aa, Homo sapiens
KRSRLLHSDYMNMTPRRPGPTRKHYQPYAPPRDFAAY

SEQ ID NO: 87 - protein, 154 aa, synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 149 aa, synthetic construct
RRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 151 aa, synthetic construct
KRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 187 aa, synthetic construct
KRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 20 aa, Picornavirus
RAEGRGSLLTCGDVEENPGP

SEQ ID NO: 92 - protein, 19 aa, Picornavirus
ATNFSLLKQAGDVEENPGP

SEQ ID NO: 93 - protein, 488 aa, synthetic construct; identical to SEQ ID NO: 46

SEQ ID NO: 94 - protein, 734 aa, synthetic construct
METDTLILWVLLLLVPGSTGDVVLTQTPPTLSATIGQSVSISCRSSQSLLHSNGNTYLHWLLQRPGQSDVLPQFLIYLVSGLGSGVPNRFSGSGSGTDFTLKISGVEAEDLGIYYCVHGTHAWTVGGGTKLELKRTVAACKPSVFIFPPSDEQLKSGTASVVCLLNNFYPREAKVQWKVDNALQSGNSQESVTEQDSKDSTYSLSSTLTCKPASIGNALLSKADYEKHKVYACEVTHQGLSSPVTKSFNRGECRAATNFSLLKQAGDVEENPGPMGWSCIILFLVATDVHATGVHSEVQLVESGGGLVQPGRSLKLSCAASGFIFSDYNMAWVRQAPKKGLEWVATIIYDGSSTNHGDDVHCHSVKGRFTISRDNAKSTLYLQMDSLRSEDTATYYCATRPGPFAYWGQGTLVTVSSASTKGPSVFPLAPSCHSKSTSGGTAALGCLVKDYFPEPVTVSWNSGALTSGVHTFPAVLQSSGLYSLSSVVTVPSSSLGTQTYICHHINGECDTMCNVNHKPSNTKVDKRVEPKSCDKTHTCPPCPKDPKFWVLVVVGGVLACYSLLVTVAFIIFWVKRGRKKBBCDLLYIFKQPFMRPVQTTQEEDGCSCRFPEEEEGGCELRVKFSRSADAPAYQQGQNQLYNELNLGRREEYCDDVLDKRRGRDPEMGGKPRRKNPQEGLYNELQKDKMAEAYSEIGMKGERRRGKGHDGLYQGLSTATKDTYDALHMQALPPR